SEQUENCE LISTING (24 sequences)

SEQ ID NO: 1 - protein, 1227 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 2 - protein, 1227 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 3 - protein, 1227 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMANSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 4 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQYNSIISVLFGTGRKEDRSVKLRITKKILEAISNLKEIPKNVAPIQEIILNVAKATKETFRQVYAGNLGAPSTLERFIAKDGQKEFDLKKLQTDLKKVIRGKSKERDWCCQEELRSYVEQNTIQYDLWAWGEMFNKAHTALKIKSTRNYNFAKQRLEQFKRIQSLNNLLVVKKLNDFFDSEFFSGEETYTICVHHLGGKRLSKLYKAWEDDPADPENAIVVLCDDLKNNFKKEPIRNILRYIFTIRQECSAQDILAAAKYNQQLDRYKSQKANPSVLGNQGFTWRNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITRISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 5 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQYNSIISVLFGTGRKEDRSVKLRITKKILEAISNLKEIPKNVAPIQEIILNVAKATKETFRQVYAGNLGAPSTLERFIAKDGQKEFDLKKLQTDLKKVIRGKSKERDWCCQEELRSYVEQNTIQYDLWAWGEMFNKAHTALKIKSTRNYNFAKQRLEQFKRIQSLNNLLVVKKLNDFFDSEFFSGEETYTICVHHLGGKRLSKLYKAWEDDPADPENAIVVLCDDLKNNFKKEPIRNILRYIFTIRQECSAQDILAAAKYNQQLDRYKSQKANPSVLGNQGFTWRNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITRISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDALARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 6 - protein, 678 aa, synthetic construct
STLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 7 - protein, 514 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPL

SEQ ID NO: 8 - protein, 116 aa, synthetic construct
ASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 9 - protein, 32 aa, synthetic construct
SGGSSGGSSGSETPGTSESATPESSGGSSGGS

SEQ ID NO: 10 - protein, 19 aa, synthetic construct
MKRTADGSEFESPKKKRKV

SEQ ID NO: 11 - protein, 17 aa, synthetic construct
KRTADGSEFEPKKKRKV

SEQ ID NO: 12 - protein, 1972 aa, synthetic construct
MKRTADGSEFESPKKKRKVKLASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYSRLQSGGSSGGSSGSETPGTSESATPESSGGSSGGSELMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMANSITGRTDVDFLISPVKNSDGIFYDSRNYEAQENAILPKNADANGAYNIARKVLWAIGQFKKAEDEKLDKVKIAISNKEWLEYAQTSVKSGGSSGGSSGSETPGTSESATPESSGGSSGGSPGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLSGGSKRTADGSEFEPKKKRKV

SEQ ID NO: 13 - protein, 1972 aa, synthetic construct
MKRTADGSEFESPKKKRKVKLASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYSRLQSGGSSGGSSGSETPGTSESATPESSGGSSGGSELMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSSGGSSGSETPGTSESATPESSGGSSGGSPGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLSGGSKRTADGSEFEPKKKRKV

SEQ ID NO: 14 - protein, 1799 aa, synthetic construct
MKRTADGSEFESPKKKRKVKLASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYSRLQSGGSSGGSSGSETPGTSESATPESSGGSSGGSELMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQYNSIISVLFGTGRKEDRSVKLRITKKILEAISNLKEIPKNVAPIQEIILNVAKATKETFRQVYAGNLGAPSTLERFIAKDGQKEFDLKKLQTDLKKVIRGKSKERDWCCQEELRSYVEQNTIQYDLWAWGEMFNKAHTALKIKSTRNYNFAKQRLEQFKRIQSLNNLLVVKKLNDFFDSEFFSGEETYTICVHHLGGKRLSKLYKAWEDDPADPENAIVVLCDDLKNNFKKEPIRNILRYIFTIRQECSAQDILAAAKYNQQLDRYKSQKANPSVLGNQGFTWRNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITRISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDALARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTSSGGSSGGSSGSETPGTSESATPESSGGSSGGSPGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLSGGSKRTADGSEFEPKKKRKV

SEQ ID NO: 15 - protein, 1799 aa, synthetic construct
MKRTADGSEFESPKKKRKVKLASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYSRLQSGGSSGGSSGSETPGTSESATPESSGGSSGGSELMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQYNSIISVLFGTGRKEDRSVKLRITKKILEAISNLKEIPKNVAPIQEIILNVAKATKETFRQVYAGNLGAPSTLERFIAKDGQKEFDLKKLQTDLKKVIRGKSKERDWCCQEELRSYVEQNTIQYDLWAWGEMFNKAHTALKIKSTRNYNFAKQRLEQFKRIQSLNNLLVVKKLNDFFDSEFFSGEETYTICVHHLGGKRLSKLYKAWEDDPADPENAIVVLCDDLKNNFKKEPIRNILRYIFTIRQECSAQDILAAAKYNQQLDRYKSQKANPSVLGNQGFTWRNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITRISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTSSGGSSGGSSGSETPGTSESATPESSGGSSGGSPGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLSGGSKRTADGSEFEPKKKRKV

SEQ ID NO: 16 - protein, 870 aa, synthetic construct
MKRTADGSEFESPKKKRKVKLASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYSRSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 17 - DNA, 23 bp, synthetic construct
  source 1..23: mol_type = other DNA
gcacatgaggatcacccatgtgc

SEQ ID NO: 18 - DNA, 503 bp, synthetic construct
  source 1..503: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccatcagtcgccggtcccaagcccggataaaatgggagggggcgggaaaccgcctaaccatgccgactgatggcagaactaagcacatgaggatcacccatgtgccgagaccttccgataggtctccaaggcacatgaggatcacccatgtgctaaattaacactgccatcagtcggcgtggactgtagaacactgccaatgccggtcccaagcccggataaaagtggagggtacagtccacgctttttttt

SEQ ID NO: 19 - DNA, 56 bp, synthetic construct
  source 1..56: mol_type = other DNA
gccatcagtcgccggtcccaagcccggataaaatgggagggggcgggaaaccgcct

SEQ ID NO: 20 - DNA, 55 bp, synthetic construct
  source 1..55: mol_type = other DNA
aacactgccaatgccggtcccaagcccggataaaagtggagggtacagtccacgc

SEQ ID NO: 21 - DNA, 21 bp, synthetic construct
  source 1..21: mol_type = other DNA
aaccatgccgactgatggcag

SEQ ID NO: 22 - DNA, 26 bp, synthetic construct
  source 1..26: mol_type = other DNA
ctgccatcagtcggcgtggactgtag

SEQ ID NO: 23 - DNA, 21 bp, synthetic construct
  source 1..21: mol_type = other DNA
taatttctactaagtgtagat

SEQ ID NO: 24 - DNA, 23 bp, synthetic construct
  source 1..23: mol_type = other DNA
agaaatccgtctttcattgacgg

(19) United States
(12) Patent Application Publication
(10) Pub. No.: US 2026/0357251 A1
(43) Pub. Date: Nov. 12, 2026

(54) CIRCULAR RNA-BASED PRIME EDITOR

(71) Applicant: INSTITUTE OF GENETICS AND DEVELOPMENTAL BIOLOGY, CHINESE ACADEMY OF SCIENCES, Beijing (CN)

(72) Inventors: Caixia GAO, Beijing (CN); Ronghong LIANG, Beijing (CN)

(21) Appl. No.: 19/485,176

(22) PCT Filed: May 16, 2024
(86) PCT No.: PCT/CN2024/093674

§ 371 Date: Nov. 17, 2025

(30) Foreign Application Priority Data
May 17, 2023 (CN) 202310555395.0

(51) Int. Cl. C12N 15/11; C12N 9/12; C12N 9/22

(52) U.S. Cl. CPC C12N 15/111; C12N 9/1276; C12N 9/226; C07K 2319/80; C07K 2319/85; C12N 2310/20; C12Y 207/07049

(57) ABSTRACT

The present invention relates to the field of genetic engineering. Specifically, the present invention relates to a prime editing system based on circular RNA for achieving site-specific and precise genome editing. More specifically, the prime editing system based on circular RNA of the present invention utilizes other nucleases or nicking enzymes than Cas9 to couple with reverse transcriptase, and realizes precise base insertions, deletions, and substitutions in target sequence in an organism's genome based on circular RNA. The invention also relates to a genetically modified and transgene-free organism produced by the method and progenies thereof.

### CROSS-REFERENCE TO RELATED APPLICATIONS

This application is a national stage application under 35 U.S.C. § 371 of International Application No. PCT/CN2024/093674, filed May 16, 2024, which claims priority to Chinese Application No. CN202310555395.0, filed May 17, 2023; the disclosures of which are expressly incorporated by reference herein.

### REFERENCE TO AN ELECTRONIC SEQUENCE LISTING

The instant application contains a Sequence Listing that has been submitted electronically and is hereby incorporated by reference in its entirety. The Sequence Listing was created on May 16, 2024, is named “PCTCN2024093674-seql.xml,” and is 38,002 bytes in size.

### TECHNICAL FIELD

The present invention relates to the field of genetic engineering. Specifically, the present invention relates to a circular RNA-dependent prime editing system for achieving site-specific and precise genome editing. More specifically, the circular RNA-dependent prime editing system of the present invention utilizes other nucleases or nicking enzymes than Cas9 to couple with reverse transcriptase, and realizes precise base insertions, deletions, and substitutions in target sequence in an organism's genome based on circular RNA. The invention also relates to a genetically modified and transgene-free organism produced by the method and progenies thereof.

### BACKGROUND

With the rapid development of biology, targeted and precise editing of organism genomes has become an urgent need for medical, agricultural and scientific research. Precise genome editing technology can treat a large number of genetic diseases in the field of medicine, create excellent agricultural traits in agriculture, and promote the development of biology. There are three main types of precise base editing systems commonly used currently: cytosine base editor (CBE), adenine base editor (ABE) and prime editor (PE). Among them, the cytosine base editor can convert cytosine (C) to uracil (U) at the target site; the adenine base editor can convert adenine (A) to hypoxanthine (I); the prime editor can realize various single base conversions and transversions, and can realize insertion, deletion and replacement of fragments. It can be seen that prime editing is more powerful, but the prime editors used nowadays only couple the reverse enzyme to Cas9 protein, which is limited by the Cas9 protein in many aspects such as target sequence selection and biological delivery. Newly discovered Cas12 series proteins: Cas12a (i.e. Cpf1), Cas12b (i.e. C2c1), Cas12c, Cas12d (i.e. CasY), Cas12e (i.e. CasX), Cas12f (i.e. Cas14), Cas12h, Cas12i, Cas12j (i.e. CasΦ), Cas12k, Cas12m and smaller proteins such as IsrB, IscB and TnpB, are structurally different from Cas9 and each has unique functional advantages. For example, Cpf1 has the advantages of high efficiency and low off-target rate; Cas12f, IsrB, IscB and TnpB proteins have the advantages of small size and easy delivery. Most of these proteins have good applications as cytosine base editors and adenine base editors when fused to base deaminases. But so far, these large numbers of newly discovered proteins have not been applied to prime editors which have more functions. Therefore, using Cas12a (i.e. Cpf1), Cas12b (i.e. C2c1), Cas12c, Cas12d (i.e. CasY), Cas12e (i.e. CasX), Cas12f (i.e. Cas14), Cas12h, Cas12i, Cas12j (i.e. CasΦ), Cas12k, Cas12m, IsrB, IscB and TnpB and other proteins to develop new prime editing systems that do not rely on Cas9 protein, will overcome the limitations of Cas9 protein, effectively utilize the advantages of other proteins, promote the application of prime editors, and play a very important role in disease treatment, agricultural trait improvement and biological research.

### BRIEF DESCRIPTION OF THE INVENTION

The present invention provides a new Circular RNA-dependent Prime Editing system (CPE, FIG. 1). According to the type of protein used in prime editing, CPE is divided into prime editing systems that use non-targeting strand nickase and rely on circular RNA (niCPE, Circular RNA-dependent Prime Editing system with nickase, FIG. 1a) and those prime editing systems that use nuclease and rely on circular RNA (nuCPE, Circular RNA-dependent Prime Editing system with nuclease, FIG. 1b). At the same time, in order to facilitate delivery, we developed a splittable sniCPE (split Circular RNA-dependent Prime Editing system with nickase, FIG. 1c) prime editing system and a snuCPE (split Circular RNA-dependent Prime Editing system with nuclease) prime editing system. In addition, we also discovered that multiple RNAs can be expressed in tandem, and based on this, we developed an efficient one-CPE (All RNA under one U6 promoter CPE) prime editing system. More importantly, we used the Cas12a-CPE system to achieve multi-gene editing in the HEK293T human cell line. The CPE prime editing system can further achieve precise genome editing of single base conversions and transversions, fragment insertions, deletions and replacements in animals, bacteria, dicots, monocots and other organisms.

### BRIEF DESCRIPTION OF THE DRAWINGS

FIG. 1: Schematic diagram of different prime editing systems.

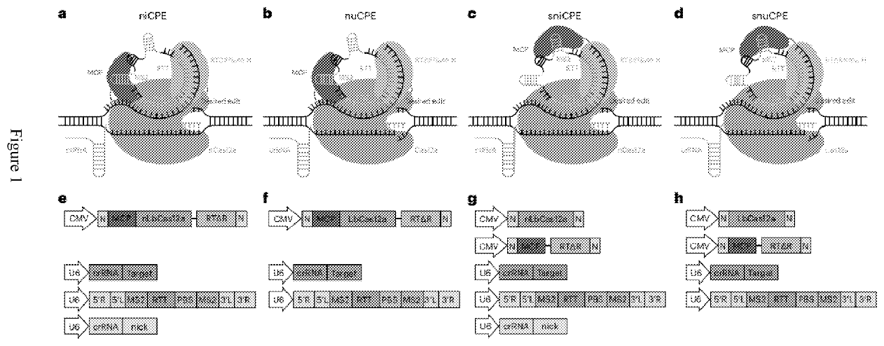

FIG. 2: Editing efficiency of different prime editing systems in copGFP reporter system.

FIG. 3: Comparison of prime editing efficiency using pegRNA and circular RNA.

FIG. 4: Editing efficiency of the Cas12a-niCPE prime editing system for different editing in various targets in human 293T cells.

FIG. 5: Editing efficiency of Cas12a-nuCPE prime editing system in human 293T cells.

FIG. 6: Editing efficiency of Cas12a-sniCPE prime editing system in human 293T cells.

FIG. 7: Editing efficiency of Cas12a-snuCPE prime editing system in human 293T cells.

FIG. 8: Comparison of editing efficiency of different prime editing systems.

FIG. 9: one-CPE boot editing system.

FIG. 10: Multi-gene prime editing system.

FIG. 11: Off-Target detection of the prime editing system CPE using circular RNA.

FIG. 12: Editing efficiency of Cas12i-nuCPE and Cas12i-niCPE prime editing systems in human 293T cells.

### DETAILED DESCRIPTION OF THE INVENTION

### I. Definition

In the present invention, unless indicated otherwise, the scientific and technological terminologies used herein refer to meanings commonly understood by a person skilled in the art. Also, the terminologies and experimental procedures used herein relating to protein and nucleotide chemistry, molecular biology, cell and tissue cultivation, microbiology, immunology, all belong to terminologies and conventional methods generally used in the art. For example, the standard DNA recombination and molecular cloning technology used herein are well known to a person skilled in the art, and are described in details in the following references: Sambrook, J., Fritsch, E. F. and Maniatis, T., Molecular Cloning: A Laboratory Manual; Cold Spring Harbor Laboratory Press: Cold Spring Harbor, 1989. In the meantime, in order to better understand the present invention, definitions and explanations for the relevant terminologies are provided below.

As used herein, the term “and/or” encompasses all combinations of items connected by the term, and each combination should be regarded as individually listed herein. For example, “A and/or B” covers “A”, “A and B”, and “B”. For example, “A, B, and/or C” covers “A”, “B”, “C”, “A and B”, “A and C”, “B and C”, and “A and B and C”.

When the term “comprise” is used herein to describe the sequence of a protein or nucleic acid, the protein or nucleic acid may consist of the sequence, or may have additional amino acids or nucleotide at one or both ends of the protein or nucleic acid, but still have the activity described in this invention. In addition, those skilled in the art know that the methionine encoded by the start codon at the N-terminus of the polypeptide will be retained under certain practical conditions (for example, when expressed in a specific expression system), but does not substantially affect the function of the polypeptide. Therefore, when describing the amino acid sequence of specific polypeptide in the specification and claims of the present application, although it may not include the methionine encoded by the start codon at the N-terminus, the sequence containing the methionine is also encompassed, correspondingly, its coding nucleotide sequence may also contain a start codon; vice versa.

“Genome” as used herein encompasses not only chromosomal DNA present in the nucleus, but also organelle DNA present in the subcellular components (e.g., mitochondria, plastids) of the cell.

As used herein, “organism” includes any organism suitable for genome editing, preferably eukaryotes. Examples of organisms include, but are not limited to, mammals such as human, mouse, rat, monkey, dog, pig, sheep, cattle, cat; poultry such as chicken, duck, goose; plants including monocots and dicots, for example, rice, corn, wheat, sorghum, barley, soybean, peanut, Arabidopsis thaliana, etc.

A “genetically modified organism” means an organism which comprises an exogenous polynucleotide or comprises a modified gene or expression regulatory sequence within its genome. For example, the exogenous polynucleotide can be stably integrated into the genome of the organism and inherited in successive generations. The exogenous polynucleotide may be integrated into the genome alone or as a part of a recombinant DNA construct. The modified gene or expression regulatory sequence is a gene or expression regulatory sequence comprising one or more nucleotide substitutions, deletions and additions in the genome of the organism.

The term “exogenous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. Although a nucleotide sequence may be represented herein as a DNA sequence (including T), when referring to RNA, one skilled in the art can readily determine the corresponding RNA sequence (i.e., substituting T with U).

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

As used herein, an “expression construct” refers to a vector suitable for expression of a nucleotide sequence of interest in an organism, such as a recombinant vector. “Expression” refers to the production of a functional product. For example, the expression of a nucleotide sequence may refer to transcription of the nucleotide sequence (such as transcribe to produce an mRNA or a functional RNA) and/or translation of RNA into a protein precursor or a mature protein.

The “expression construct” of the present invention can be a linear nucleic acid fragment, a circular plasmid, a viral vector, or, in some embodiments, an RNA capable of translation (such as mRNA), such as RNA generated by in vitro transcription. For use in mammals such as humans, the expression construct is preferably a viral vector, such as an adeno-associated virus (AAV), lentivirus, adenoviral vector, most preferably an AAV vector.

“Expression construct” of the invention may comprise regulatory sequences and nucleotide sequences of interest that are derived from different sources, or regulatory sequences and nucleotide sequences of interest derived from the same source, but arranged in a manner different than that normally found in nature.

“Promoter” refers to a nucleic acid fragment capable of controlling the transcription of another nucleic acid fragment. In some embodiments of the present invention, the promoter is a promoter capable of controlling the transcription of a gene in a cell, whether or not it is derived from the cell. The promoter may be a constitutive promoter or a tissue-specific promoter or a developmentally-regulated promoter or an inducible promoter.

Examples of promoters that can be used in the present invention include but are not limited to polymerase (pol) I, pol II or pol III promoters. Examples of pol I promoters include chicken RNA pol I promoter. Examples of pol II promoters include but are not limited to cytomegalovirus immediate early (CMV) promoter, rous sarcoma virus long terminal repeat (RSV-LTR) promoter and simian virus 40 (SV40) immediate early promoter. Examples of pol III promoters include U6 and H1 promoter. Inducible promoter such as metalothionein promoter can be used. Other examples of promoters include T7 bacteriophage promoter, T3 bacteriophage promoter, β-galactosidase promoter and Sp6 bacteriophage promoter etc. When used for plants, promoters that can be used include but are not limited to cauliflower mosaic virus 35S promoter, maize Ubi-1 promoter, wheat U6 promoter, rice U3 promoter, maize U3 promoter and rice actin promoter etc.

“Introduction” of a nucleic acid molecule (e.g., plasmid, linear nucleic acid fragment, RNA, etc.) or protein into an organism means that the nucleic acid or protein is used to transform a cell of the organism such that the nucleic acid or protein functions in the cell. As used in the present invention, “transformation” includes both stable and transient transformations. “Stable transformation” refers to the introduction of an exogenous nucleotide sequence into the genome, resulting in the stable inheritance of foreign genes. Once stably transformed, the exogenous nucleic acid sequence is stably integrated into the genome of the organism and any of its successive generations. “Transient transformation” refers to the introduction of a nucleic acid molecule or protein into a cell, performing its function without the stable inheritance of an exogenous gene. In transient transformation, the exogenous nucleic acid sequence is not integrated into the genome.

“Trait” refers to the physiological, morphological, biochemical, or physical characteristics of a cell or an organism.

### II. Prime Editing System

In one aspect, the present invention provides a prime editing system for targeted modification of genomic DNA sequence of an organism, which includes:
i) a) a CRISPR nuclease and/or an expression construct containing a nucleotide sequence encoding said CRISPR nuclease, and a reverse transcriptase and/or an expression construct containing a nucleotide sequence encoding said reverse transcriptase, or
b) a prime editing fusion protein and/or an expression construct containing a nucleotide sequence encoding the prime editing fusion protein, wherein the prime editing fusion protein comprises a CRISPR nuclease and a reverse transcriptase;
ii) an editing guide RNA (gRNA) directed to a target sequence in the genomic DNA and/or an expression construct containing a nucleotide sequence encoding said editing gRNA; and
iii) a circular RNA containing a reverse transcription template (RTT) sequence and a primer binding site (PBS) sequence and/or an expression construct containing a nucleotide sequence encoding said circular RNA,
preferably, the CRISPR nuclease is not a Cas9 nuclease or a variant thereof.

As used herein, a “prime editing system” refers to the combination of components required for reverse transcription-based genome editing of a genome in a cell. Each component of the system, such as CRISPR nuclease, reverse transcriptase, prime editing fusion protein, gRNA, circular RNA or their expression constructs, etc., can exist independently, or can be used in any combination as a composition. The individual components of the system can be packaged independently or together into viruses, virus-like particles, virions, liposomes, vesicles, exosomes, liposomal nanoparticles (LNPs), and the like.

The CRISPR nuclease of the invention may be a CRISPR nuclease other than Cas9 nuclease or a variant thereof. Examples of CRISPR nucleases suitable for the present invention include, but are not limited to, Cas12a (i.e., Cpf1), Cas12b (i.e., C2c1), Cas12c, Cas12d (i.e., CasY), Cas12e (i.e., CasX), Cas12f (i.e., Cas14), Cas12h, Cas12i, Cas12j (i.e., CasΦ), Cas12k, Cas12m, IsrB, IscB and TnpB. In some specific embodiments, the CRISPR nuclease is Cas12a. In other specific embodiments, the CRISPR nuclease is Cas12i.

In some embodiments, the CRISPR nuclease also includes the nickase form thereof. CRISPR nuclease in the form of nickase may also be referred to herein as a CRISPR nickase, which forms a nick in only one strand of a double-stranded nucleic acid molecule but does not completely cut the double-stranded nucleic acid. In some embodiments, the CRISPR nickase only nicks the complementary strand (also called the non-targeting strand in the art) of the strand where the target sequence is located in the double-stranded nucleic acid molecule.

As used herein, a “target sequence” refers to a sequence of approximately 20 nucleotides in length in the genome that is characterized by a 5′ or 3′ flanking PAM (protospacer adjacent motif) sequence. Generally, PAM is required for the CRISPR nuclease or variants thereof and the guide RNA to recognize the target sequence and form a complex. For example, for Cas12a nuclease and variants thereof, the target sequence is immediately adjacent to a PAM, such as 5′-TTTV-3′, at its 3′ end. Based on the presence of PAMs, those skilled in the art can easily determine target sequences in the genome that can be used for targeting. And depending on the position of the PAM, the target sequence can be located on any strand of the genomic DNA molecule. The strand bound by crRNA is called the targeting strand, and the strand complementary to the targeting strand is called the non-targeting strand. For Cas12a or its derivatives such as Cas12a nickase, the target sequence is preferably 20 to 23 nucleotides in length. Depending on the different CRISPR nuclease or its different variants, the PAM sequence will differ.

In some specific embodiments, the CRISPR nuclease is Cas12a, which comprises an amino acid sequence shown in SEQ ID NO: 1 or 2. In some preferred embodiments, the CRISPR nuclease is Cas12a nickase, in which the amino acid R at position 1138 is substituted with respect to SEQ ID NO: 1 or 2, for example, the amino acid R at position 1138 is substituted by A. In some preferred embodiments, the CRISPR nuclease comprises the amino acid sequence shown in SEQ ID NO:3.

In some specific embodiments, the CRISPR nuclease is Cas12i, which comprises the amino acid sequence set forth in SEQ ID NO:4. In some preferred embodiments, the CRISPR nuclease is Cas12i nickase in which amino acid W at position 854 is substituted relative to SEQ ID NO: 4, for example, amino acid W at position 854 is substituted by A. In some preferred embodiments, the Cas12i nickase comprises the amino acid sequence shown in SEQ ID NO:5.

The reverse transcriptase of the present invention can be derived from different sources. In some embodiments, the reverse transcriptase is a virus-derived reverse transcriptase. For example, in some embodiments, the reverse transcriptase is M-MLV reverse transcriptase. An exemplary wild-type M-MLV reverse transcriptase has the amino acid sequence shown in SEQ ID NO: 6.

In some embodiments, the RNase H domain of the reverse transcriptase, such as M-MLV reverse transcriptase, is mutated or deleted. In some embodiments, the M-MLV reverse transcriptase in which the RNase H domain is mutated or deleted comprises the amino acid sequence shown in SEQ ID NO:7.

In some embodiments, the reverse transcriptase and/or the prime editing fusion protein of the present invention may further comprise one or more RNA aptamer binding protein sequences. In some embodiments, the reverse transcriptase of the invention further comprises one or more RNA aptamer binding protein sequences. In some embodiments, the prime editing fusion protein of the present invention further comprises one or more RNA aptamer binding protein sequences.

“RNA aptamer binding protein” refers to a protein that can specifically bind to an “RNA aptamer” with a specific sequence and structure. Proteins containing one or more RNA aptamer binding protein sequences in a cell can thus be recruited to nucleic acid molecules containing the corresponding RNA aptamer, and vice versa. Many such combinations of “RNA aptamer binding proteins” and “RNA aptamers” capable of specific binding are known in the art.

In some embodiments, the RNA aptamer binding protein is an MCP protein and the corresponding RNA aptamer is MS2. MCP protein can specifically bind to MS2 or multiple MS2s in tandem. An exemplary MCP comprises the amino acid sequence set forth in SEQ ID NO: 8. An exemplary MS2 comprises the nucleotide sequence set forth in SEQ ID NO:17.

As used herein, a “linker” may be a non-functional amino acid sequence which is 1-50 or more (e.g., 1, 2, 3, 4, 5, 6, 7, 8, 9, 10, 11, 12, 13, 14, 15, 16, 17, 18, 19, 20 or 20-25, 25-50) amino acids in length and has no secondary or higher structures. For example, the linker may be a flexible linker. In some specific embodiments, the linker comprises the amino acid sequence set forth in SEQ ID NO:9.

In some embodiments of the invention, the CRISPR nuclease, reverse transcriptase, or prime editing fusion protein of the invention may also comprises one or more nuclear localization sequences (NLS). Generally, the one or more NLSs in the CRISPR nuclease, reverse transcriptase or prime editing fusion protein should be strong enough to drive accumulation of the CRISPR nuclease, reverse transcriptase or prime editing fusion protein in the nucleus of the cell in amounts that enable its gene-editing function. Generally, the strength of nuclear localization activity is determined by the number and location of NLS(s) in the CRISPR nuclease, reverse transcriptase or prime editing fusion protein, the specific NLS(s) used, or a combination of these factors. An exemplary NLS may comprise an amino acid sequence set forth in SEQ ID NO: 10 or 11.

However, depending on the DNA location to be edited, the CRISPR nuclease, reverse transcriptase, or prime editing fusion protein of the present invention may also comprise other localization sequences, such as cytoplasmic localization sequences, chloroplast localization sequences, mitochondrial localization sequences, etc.

In some embodiments, the prime editing fusion protein comprises an amino acid sequence selected from SEQ ID NO: 12-15, preferably comprises the amino acid sequence of SEQ ID NO: 12.

In some embodiments, the prime editing system comprises:
i) a prime editing fusion protein and/or an expression construct containing a nucleotide sequence encoding said prime editing fusion protein, wherein said prime editing fusion protein comprises an amino acid sequence selected from SEQ ID NOs: 12-15, preferably comprises the amino acid sequence of SEQ ID NO: 12;
ii) an editing guide RNA (gRNA) and/or an expression construct containing a nucleotide sequence encoding said editing gRNA; and
iii) a circular RNA containing a reverse transcription template (RTT) sequence and a primer binding site (PBS) sequence and/or an expression construct containing a nucleotide sequence encoding said circular RNA.

In some embodiments, the prime editing system comprises:
i) a CRISPR nuclease and/or an expression construct containing a nucleotide sequence encoding said CRISPR nuclease, and a reverse transcriptase and/or an expression construct containing a nucleotide sequence encoding said reverse transcriptase;
ii) an editing guide RNA (gRNA) and/or an expression construct containing a nucleotide sequence encoding said editing gRNA; and
iii) a circular RNA containing a reverse transcription template (RTT) sequence and a primer binding site (PBS) sequence and/or an expression construct containing a nucleotide sequence encoding said circular RNA,
wherein the CRISPR nuclease comprises an amino acid sequence selected from SEQ ID NO: 1-5, preferably the amino acid sequence of SEQ ID NO: 3 or 5,
wherein the reverse transcriptase comprises the amino acid sequence shown in SEQ ID NO: 16.

In order to obtain effective expression in different organisms, in the present invention, the nucleotide sequence encoding the CRISPR nuclease, reverse transcriptase, or prime editing fusion protein can be codon-optimized for the organism species whose genome is to be modified.

Codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000).

The guide RNA (gRNA) of the present invention refers to a gRNA that is compatible with the CRISPR nuclease. gNRA may typically comprise a scaffold sequence and a guide sequence (also called a seed sequence or a spacer sequence). The guide sequence is configured to have sufficient sequence identity (preferably 100% identity) with the target sequence, so that it can bind to the complementary strand of the target sequence through base pairing to achieve sequence-specific targeting. The scaffold sequence depends on the CRISPR nuclease as used. For example, for Cas12a such as LbCas12a, the scaffold sequence of the gRNA may comprise the nucleotide sequence shown in SEQ ID NO: 23. For Cas12i, the scaffold sequence of its gRNA may comprise the nucleotide sequence shown in SEQ ID NO: 24. CRISPR nuclease can bind to the scaffold sequence of gRNA to form a CRISPR nuclease-gRNA complex, and is targeted to the genome target sequence under the guidance of the guide sequence to exert its nuclease or nickase function. Those skilled in the art can design appropriate gRNAs for different CRISPR nucleases and different target sequences. The guide RNA directed against the target sequence to be edited is called editing guide RNA (editing gRNA) in the present invention.

The “circular RNA” of the present invention contains a reverse transcription template (RTT) sequence and a primer binding site (PBS) sequence directly linked (RTT-PBS sequence), where the RTT sequence is located to the 5′ end of the PBS sequence.

In some embodiments, the circular RNA further comprises one or more (e.g., 1 or 2 or 3 or more) RNA aptamer sequences, such as MS2 sequences. The one or more RNA aptamer sequences in the circular RNA can be used to recruit the reverse transcriptase comprising an RNA aptamer binding protein in the cell, or to allow the circular RNA to be recruit to the CRISPR nuclease-gRNA-genomic DNA complex.

In some embodiments, the primer binding site sequence is configured to be complementary to at least a portion of the target sequence (preferably perfectly paired with at least a portion of the target sequence). Preferably, the primer binding site sequence is complementary to at least a portion of the 3′ free single strand caused by the nick in the DNA strand where the target sequence is located (preferably completely paired with at least a part of the 3′ free single strand), especially is complementary to the nucleotide sequence at 3′ end of the 3′ free single strand (preferably perfectly paired). When the 3′ free single strand of the strand binds to the primer binding sequence through base pairing, the 3′ free single strand can serve as a primer, using the reverse transcription template sequence immediately adjacent to the primer binding site sequence as a template, reverse transcription is performed under the action of the reverse transcriptase in the prime editing fusion protein or the reverse transcriptase recruited through the RNA aptamer binding protein, and the DNA sequence corresponding to the reverse transcription template (RTT) sequence is extended.

The sequence of the primer binding site depends on the length of the free single strand, formed by the CRISPR nuclease as used, in the strand where the target sequence is located, however, it should have a minimum length to ensure specific binding. In some embodiments, the primer binding site sequence may be 4-20 nucleotides in length, for example, 4, 5, 6, 7, 8, 9, 10, 11, 12, 13, 14, 15, 16, 17, 18, 19, 20 nucleotides in length.

The RTT sequence can be any sequence. Through the above-mentioned reverse transcription, its sequence information can be integrated into the DNA strand where the target sequence is located (that is, the strand containing the target sequence PAM), and then through the DNA repair function of the cell, a double-stranded DNA containing the RTT sequence information can be formed. In some embodiments, the RTT sequence contains the desired modification. For example, the desired modifications include substitutions, deletions, and/or additions of one or more nucleotides. In some embodiments, the RTT sequence is configured to correspond to the sequence downstream of the nick of the target sequence (e.g., complementary to at least a portion of the sequence downstream of the nick of the target sequence) but comprise the desired modifications. The desired modifications include substitutions, deletions and/or additions of one or more nucleotides.

In some embodiments, the prime editing system further comprises a nicking gRNA (the nicking gRNA is used for generating an additional nick) and/or an expression construct comprising a nucleotide sequence encoding the nicking gRNA, wherein the nicking gRNA comprises a guide sequence and a scaffold sequence. In some preferred embodiments, the nicking gRNA does not contain the reverse transcription (RT) template sequence and the primer binding site (PBS) sequence.

The guide sequence (also referred to as seed sequence or spacer sequence) in the nicking gRNA of the present invention is configured to have sufficient sequence identity (preferably 100% identity) to a nicking target sequence in the genome, so that the fusion protein of the present invention is targeted to the nicking target sequence, resulting in a nick in the nicking target sequence, wherein the nicking target sequence and the target sequence (editing gRNA target sequence) targeted by the editing gRNA (target sequence to be edited) are located on the opposite strand of the genome DNA. In some embodiments, the nick formed by the nicking RNA and the nick formed by the editing gRNA are about 1 to about 300 or more nucleotides apart, e.g., 1, 2, 3, 4, 5, about 10, about 20, about 30, about 40, about 50, about 75, about 100, about 125, about 150, about 175, about 200, about 225, about 250, about 275, about 300 nucleotides or more nucleotides apart. In some embodiments, the nick created by the nicking RNA is located upstream or downstream of the nick formed by the editing gRNA (upstream or downstream refers to the DNA strand on which the editing gRNA target sequence is located). In some embodiments, the guide sequence in the nicking gRNA has sufficient sequence identity (preferably 100% identity) to the editing gRNA target sequence on the opposite strand after editing event occur (modified editing gRNA target sequence), so that the nicking gRNA targets only the nicking target sequence that is generated after editing gRNA-induced sequence targeting and modification. In some embodiments, the PAM of the nicking target sequence is located within the complementary sequence of the editing gRNA target sequence.

In some embodiments, the prime editing system comprises a plurality (e.g., 2, 3, 4, 5 or more) of editing gRNAs and/or an expression construct contains a nucleotide sequence encoding the plurality of editing gRNAs.

Accordingly, in some embodiments, the circular RNA comprises multiple sets (e.g., 2, 3, 4, 5 or more) of RTT-PBS sequences in tandem corresponding to the plurality of editing gRNAs, each set of RTT-PBS sequences includes a reverse transcription template (RTT) sequence and a primer binding site (PBS) sequence which are directly linked, and the RTT sequence is located at the 5′ end of the PBS sequence. In some embodiments, the multiple editing gRNAs target different target sequences, and the corresponding sets of RTT-PBS sequences are used to introduce different modifications, so that the prime editing system can target and modify different target sequences.

The “expression construct containing a nucleotide sequence encoding the circular RNA” of the present invention may also contain elements required for realizing RNA circularization. The components can be referred to Jacob L. Litke et al., Nature Biotechnology, Vol 37, June 2019, 667-675, or Wesselhoeft A. R. et al., (2018) Engineering circular RNA for potent and stable translation in eukaryotic cells. Other elements known in the art that can achieve RNA circularization may also be used in the present invention.

In some embodiments, the “expression construct containing a nucleotide sequence encoding the circular RNA” includes coding sequences of the following: 5′-first ribozyme-first circularization arm-RTT-PBS-second circularization arm-second ribozyme-3′. When transcribed into RNA in cells, the first ribozyme and the second ribozyme can self-cleave to produce “5′-first circularization arm-RTT-PBS-second circularization arm-3”, and the first circularization arm and the second circularization arm can be linked to each other to form a circular RNA containing RTT and PBS. In some embodiments, the RTT-PBS may also include one or more RNA aptamer sequences, such as MS2 sequences, on either or both sides.

In some specific embodiments, the first ribozyme comprises the nucleotide sequence set forth in SEQ ID NO:19. In some specific embodiments, the second ribozyme comprises the nucleotide sequence set forth in SEQ ID NO:20. In some specific embodiments, the first circularization arm comprises the nucleotide sequence set forth in SEQ ID NO:21. In some specific embodiments, the second circularization arm comprises the nucleotide sequence set forth in SEQ ID NO:22.

In some embodiments, the editing gRNA and the circular RNA and optionally the nicking gRNA are encoded by a same expression construct. In some embodiments, the expression construct comprises (e.g., from the 5′ to 3′ direction) a nucleotide sequence encoding the circular RNA, a nucleotide sequence encoding the editing gRNA, and optionally a nucleotide sequence encoding the nicking gRNA. In some embodiments, expression (transcription) of the nucleotide sequence encoding the circular RNA, the nucleotide sequence encoding the editing gRNA, and optionally the nucleotide sequence encoding the nicking gRNA is driven by the same promoter, such as the U6 promoter. Due to the RNA cleavage activity of CRISPR nuclease/nickase for crRNA maturation, RNA molecules transcribed from the same promoter can generate separate circular RNA, editing gRNA, and optional nicking gRNA.

In another aspect, the invention provides a Cas12a nickase and/or an expression construct comprising its coding sequence, wherein the amino acid at position 1138 is substituted relative to SEQ ID NO: 1 or 2, for example, the amino acid R at position 1138 is replaced by A. In some preferred embodiments, the Cas12a nickase comprises the amino acid sequence shown in SEQ ID NO: 3.

In another aspect, the invention provides a Cas12i nickase and/or an expression construct comprising a coding sequence thereof, in which the amino acid at position 854 is substituted relative to SEQ ID NO: 4, for example, amino acid W at position 854 is replaced by A. In some preferred embodiments, the Cas12i nickase comprises the amino acid sequence shown in SEQ ID NO: 5.

In another aspect, the invention provides the use of the Cas12a nickase or Cas12i nickase of the invention, or an expression construct comprising the coding sequence thereof, in gene editing, such as prime editing.

### III. Methods to Modify a Target Sequence in the Genome of a Cell

In another aspect, the invention provides a method of producing a genetically modified cell, comprising introducing a prime editing system of the invention into at least one of said cells, thereby resulting in modification of the genomic sequence of said at least one cell.

The modification comprises substitution, deletion and/or addition of one or more nucleotide. For example, the modification comprises one or more substitutions selected from: C to T substitution, C to G substitution, C to A substitution, G to T substitution, G to C substitution, G to A substitution, A to T substitution, A to G substitution, A to C substitution, T to C substitution, T to G substitution and T to A substitution; and/or comprises deletion of one or more nucleotides, for example, deletion of 1 to about 100 or more nucleotides, e.g. deletion of 1, 2, 3, 4, 5, about 10, about 20, about 30, about 40, about 50, about 75, about 100 nucleotides; and/or comprises insertion of one or more nucleotides, for example, insertion of 1 to about 100 or more nucleotides, for example, insertion of 1 to about 100 or more nucleotides, for example, insertion of 1, 2, 3, 4, 5, about 10, about 20, about 30, about 40, about 50, about 75, about 100 nucleotides. The modification may be located in or near the target sequence of the gRNA, for example downstream of the target sequence.

In another aspect, the invention also provides a method of producing a genetically modified cell, comprising introducing the prime editing system of the invention into the cell.

In another aspect, the invention also provides a genetically modified organism comprising a genetically modified cell produced by the method of the invention or progeny cells thereof.

In the present invention, the modification can be located anywhere in the genome, such as located within a functional gene such as a protein-coding gene, or located in a gene expression regulatory region such as a promoter region or an enhancer region, thereby achieving functional modification of the gene or modification of gene expression. Modifications in the genomic sequence of the cell can be detected by T7EI, PCR/RE or sequencing methods.

In the method of the present invention, the prime editing system can be introduced into the cell through various methods well known to those skilled in the art.

Methods that can be used to introduce the prime editing system of the present invention into the cell include, but are not limited to: calcium phosphate transfection, protoplast fusion, electroporation, lipofection, microinjection, viral infection (such as baculovirus, vaccinia virus, adenovirus viruses, adeno-associated viruses, lentiviruses and other viruses), biolistics, PEG-mediated protoplast transformation, Agrobacterium-mediated transformation.

The cell that can be gene edited by the method of the present invention can be from, for example, prokaryotes such as bacteria; eukaryotes including: mammals such as human, mouse, rat, monkey, dog, pig, sheep, cattle, cat; poultry such as chicken, duck, goose; plants, including monocots and dicots, such as rice, corn, wheat, sorghum, barley, soybeans, peanuts, Arabidopsis, etc. In some preferred embodiments, the cells are from human.

In some embodiments, the method of the invention is performed in vitro. For example, the cell is an isolated cell, or a cell in an isolated tissue or organ.

In other embodiments, the method of the present invention can also be performed in vivo. For example, the cell is a cell in an organism, and the system of the present invention can be introduced into the cell in vivo by, for example, virus- or Agrobacterium-mediated methods.

### IV. Method for Producing a Genetically Modified Plant

In another aspect, the present invention provides a method of producing a genetically modified plant, comprising the step of introducing the prime editing system of the present invention into at least one plant, resulting in modification in genome of the at least one plant. The modification comprises substitution, deletion and/or addition of one or more nucleotide. For example, the modification comprises one or more substitutions selected from: C to T substitution, C to G substitution, C to A substitution, G to T substitution, G to C substitution, G to A substitution, A to T substitution, A to G substitution, A to C substitution, T to C substitution, T to G substitution and T to A substitution; and/or comprises deletion of one or more nucleotides, for example, deletion of 1 to about 100 or more nucleotides, e.g. deletion of 1, 2, 3, 4, 5, about 10, about 20, about 30, about 40, about 50, about 75, about 100 nucleotides; and/or comprises insertion of one or more nucleotides, for example, insertion of 1 to about 100 or more nucleotides, for example, insertion of 1 to about 100 or more nucleotides, for example, insertion of 1, 2, 3, 4, 5, about 10, about 20, about 30, about 40, about 50, about 75, about 100 nucleotides.

In some embodiments, the method further comprises the step of screening a plant with the desired modification from the at least one plant.

In the method of the present invention, the prime editing system may be introduced into the plants by a variety of methods well known to those skilled in the art. The methods used for introducing the prime editing system of the present invention into a plant include, but are not limited to a gene gun method, PEG-mediated protoplast transformation, Agrobacterium-mediated transformation, plant virus-mediated transformation, pollen tube pathway and ovary injection method. Preferably, the genome editing system is introduced into the plant by transient transformation.

In the method of the present invention, modification of the genome may be achieved by only introducing or producing the fusion protein and the gRNAs in the plant cells, and the modification can be stably inherited without stably transforming exogenous polynucleotides encoding the components of the prime editing system into the plant. This avoids the potential off-target effects due to the stably existing (continuously produced) prime editing system and also avoids the integration of the exogenous nucleotide sequences in the plant genome, thereby providing greater biosafety.

In some preferred embodiments, the introduction is carried out in the absence of selection pressure to avoid integration of the exogenous nucleotide sequence into the plant genome.

In some embodiments, the introduction comprises transforming the prime editing system of the present invention into an isolated plant cell or tissue and then regenerating the transformed plant cell or tissue into an intact plant. Preferably, the regeneration is carried out in the absence of selection pressure, i.e., no selection agent for the selectable gene on the expression vector is used during tissue culture. Avoiding the use of a selection agent can increase the regeneration efficiency of the plant, obtaining a modified plant free of exogenous nucleotide sequences.

In other embodiments, the prime editing system of the present invention can be transformed into specific parts of an intact plant, such as leaves, shoot tips, pollen tubes, young ears or hypocotyls. This is particularly suitable for the transformation of plants that are difficult to regenerate in tissue culture.

In some embodiments of the invention, the in vitro expressed protein and/or the in vitro transcribed RNA molecule (e.g., the expression construct is in vitro transcribed RNA molecule) are directly transformed into the plant. The protein and/or RNA molecule is capable of performing prime editing in plant cells and is subsequently degraded by the cell, avoiding integration of the exogenous nucleotide sequence in the plant genome.

Thus, in some embodiments, using the method of the present invention for plant genetic modification and breeding can obtain plants with the genome free of exogenous polynucleotide integration, namely transgene-free modified plants.

In some embodiments of the present invention, the modified genome region is associated with plant traits, such as the agronomic traits, so that modification results in a plant having altered (preferably improved) traits, such as agronomic traits, relative to a wild type plant.

In some embodiments, the method further comprises the step of screening a plant having the desired modification and/or desired traits, such as agronomic traits.

In some embodiments of the present invention, the method further comprises the step of obtaining progenies of the genetically modified plant. Preferably, the genetically modified plant or the progenies thereof have the desired modification and/or the desired traits, such as agronomic traits.

In another aspect, the invention also provides a genetically modified plant or progenies or a part thereof, wherein the plant is obtained by the method according to the present invention as described above. In some embodiments, the genetically modified plant or the progenies or a part thereof is transgene-free. Preferably, the genetically modified plant or progenies thereof have the desired genetic modification and/or the desired traits, such as agronomic traits.

In another aspect, the present invention provides a method of plant breeding comprising crossing a first genetically modified plant obtained by the above method of the present invention with a second plant not containing the modification, thereby the genetic modification is introduced into the second plant. Preferably, the first genetically modified plant has the desired traits, such as the agronomic traits.

“Agronomic trait” is a measurable parameter including but not limited to, leaf greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, disease resistance, cold resistance, salt tolerance, and tiller number and so on.

### V. Therapeutic Use

The present invention also covers the use of the prime editing system of the present invention in disease treatment.

By modifying disease-related genes through the prime editing system of the present invention, the up-regulation, down-regulation, inactivation, activation or mutation correction of disease-related genes can be achieved, thereby achieving prevention and/or treatment of diseases. For example, the genome modification described in the present invention can be located in the protein coding region of a disease-related gene, or can be located in a gene expression regulatory region such as a promoter region or enhancer region, thereby achieving functional modification or expression modification of the disease-related gene. Therefore, modification of disease-related genes described herein includes modifications to the disease-related genes themselves (such as protein coding regions), as well as modifications to their expression regulatory regions (such as promoters, enhancers, introns, etc.).

A “disease-related” gene refers to any gene that produces a transcription or translation product at abnormal levels or in an abnormal form in cells derived from disease-affected tissue as compared to non-disease control tissues or cells. Where altered expression is associated with the emergence and/or progression of a disease, it may be a gene that is expressed at an abnormally high level; or it may be a gene that is expressed at an abnormally low level. Disease-related genes also refer to genes that have one or more mutations or genetic variants that are directly responsible for or in linkage disequilibrium with one or more genes responsible for the etiology of the disease. The mutation or genetic variation is, for example, a single nucleotide variation (SNV). The products of transcription or translation may be known or unknown, and may be at normal or abnormal levels.

Accordingly, the invention also provides a method of treating a disease in a subject in need thereof, comprising delivering to said subject an effective amount of the prime editing system of the invention to modify a gene related to said disease. The invention also provides the use of the prime editing system for the preparation of a pharmaceutical composition for treating a disease in a subject in need thereof, wherein the prime editing system is used to modify a gene related to said disease. The present invention also provides a pharmaceutical composition for treating a disease in a subject in need thereof, comprising the prime editing system of the present invention, and optionally a pharmaceutically acceptable carrier, wherein the prime editing system is used to modify a gene related to said disease.

Preferably, the “subject” of the present invention is a mammal, such as a human.

In some embodiments, the prime editing system described herein is used to introduce a point mutation into a nucleic acid.

In some embodiments, the prime editing system described herein is used for the correction of genetic defects, such as the correction of point mutations that result in loss of function in a gene product. In some embodiments, the genetic defect is associated with a disease or condition (e.g., a lysosomal storage disease or a metabolic disease, such as, for example, Type I diabetes). In some embodiments, the method provided herein can be used to introduce an inactivating point mutation into a gene or allele encoding a gene product associated with a disease or disorder.

In some embodiments, the embodiments described herein are intended for the treatment of patients with diseases associated with or caused by point mutations that can be corrected by the prime editing system provided herein. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease.

In some embodiments, the embodiments described herein are intended to be useful in the treatment of mitochondrial diseases or disorders. As used herein, “mitochondrial disease” refers to diseases caused by abnormal mitochondria, such as mitochondrial gene mutations, enzymatic pathways, etc. Examples of disorders include, but are not limited to: neurological disorders, loss of motor control, muscle weakness and pain, gastrointestinal disorders and difficulty swallowing, poor growth, heart disease, liver disease, diabetes, respiratory complications, epilepsy, vision/hearing problems, lactic acidosis, developmental delay, and susceptibility to infection.

Examples of diseases described in the present invention include, but are not limited to, genetic diseases, circulatory system diseases, muscle diseases, brain, central nervous system and immune system diseases, Alzheimer's disease, secretase disorders, amyotrophic lateral sclerosis (ALS)), autism, trinucleotide repeat expansion disorders, hearing disorders, gene-targeted therapy of non-dividing cells (neurons, muscles), liver and kidney diseases, epithelial cell and lung diseases, cancer, Usher syndrome or retinitis pigmentosa-39, cystic fibrosis, HIV and AIDS, beta thalassemia, sickle cell disease, herpes simplex virus, autism, drug addiction, age-related macular degeneration, schizophrenia. Other diseases treated by correcting point mutations or introducing inactivating mutations into disease-related genes are known to those skilled in the art, and thus the present disclosure is not limited in this regard. In addition to the diseases illustratively described in the present invention, the strategies and prime editing systems provided by the present invention can also be used to treat other related diseases, and these uses will be obvious to those skilled in the art. For diseases or targets applicable to the present invention, reference can be made to WO2015089465A1 (PCT/US2014/070135), WO2016205711A1 (PCT/US2016/038181), WO2018141835A1 (PCT/EP2018/052491), WO2020191234A1 (PCT/US2 020/023713), WO2020191233A1 (PCT/US2020/023712), WO2019079347A1 (PCT/US2018/056146), and WO2021155065A1 (PCT/US2021/015580).

Administration of the prime editing system or pharmaceutical composition of the invention can be tailored to the weight and species of the patient or subject. The frequency of administration is within the limits permitted by medical or veterinary medicine. It depends on general factors including the patient or subject's age, sex, general health, other conditions, and the specific condition or symptom being addressed.

### VI. Kit

The invention also includes a kit for use in the method of the invention, the kit comprising the components of the prime editing system of the invention. The kit may also contain reagents for introducing the prime editing system into an organism or cells of an organism. The kit generally includes labels indicating the intended use and/or method of use of the contents of the kit. The term label includes any written or recorded material on or provided with the kit or otherwise provided with the kit.

### EXAMPLES

### Materials and Methods

### 1. Vector Construction

The vectors were constructed by using the available prime editor vector backbones in the laboratory. The main vectors constructed are:

niCPE:
Cas12a-niCPE
Cas12a-sniCPE
Cas12i-niCPE
   nuCPE:
Cas12a-nuCPE
Cas12a-snuCPE
Cas12i-nuCPE

### CirRNA:

5′+3′MS2-CirRNA

### Guide RNA:

crRNA (Cas12a)
crRNA (Cas12i)

### 2. Human HEK293T Cell Culture and Transformation

### 2.1 Thawing the Cells

(1) the HEK293T cell line was taken out of the liquid nitrogen and heated in a 37 °C water bath for 2 minutes for thawing.
(2) the cells were removed from the cryovial and slowly added to a 15 mL centrifuge tube containing 10 mL of culture medium.
(3) Centrifuged at 500×g for 3 minutes to pellet cells and supernatant was removed.
(4) 10 mL of culture medium was added to the pellet, transferred to a T175 culture bottle, and placed in a 37 °C incubator containing 5% CO2.
(5) the culture medium was replaced after 24 hours and culture was continued in a 37 °C incubator containing 5% CO2.

### 2.2 Culturing the Cells

(1) the cell culture medium was carefully aspirated out from the T175 culture flask.
(2) the cells were gently washed with 10 mL PBS solution.
(3) the cells were treated with 2 mL of trypsin: the T175 cell culture flask was placed in a 37 °C incubator for 2 minutes to allow trypsin to fully dissociate the cells.
(4) 10 mL of cell culture medium was added to the T175 cell culture flask to resuspend the cells and inactivate trypsin.
(5) the cell suspension was pipetted up and down with a 10 mL pipette to obtain a single-cell suspension.
(6) After diluting the cells at a ratio of 1:10, culture was continued in a 37 °C incubator containing 5% CO2.

### 2.3 Plating the Cells

(1) 15 μL of single cell suspension from 2.1-(5) was mixed with 15 μL of trypan blue, and placed at room temperature for 1-2 minutes.
(2) a new cell counter was used, and 10 μL of cell and trypan blue mixture was added to each of the A and B counting tanks, the counting plate was inserted into the cell counter and the counts were read.
(3) the required dilution factor was calculated so that the final plating amount is 45,000 cells in 250 μL of cell culture medium per well in a 48-well plate. Dilution factor=(A count+B count)/(2×4×45000).
(4) the cells were diluted according to the dilution factor, and an adjustable-width multi-channel pipette was used to distribute the cell suspension into a 48-well plate. 250 μL of cell suspension was added to each well.
(5) the 48-well plate containing cells was placed in a 37 °C incubator and cultured for 16 to 24 hours for liposome transformation.

### 2.4 Transfected Cells

(1) Before transfecting cells, the growth status of the cells was observed under a microscope, preferably at 60% coverage.
(2) 300 ng protein expression plasmid and 100 ng RNA expression plasmid were mixed in Opti-MEM to make the total volume 12.5 μL.
(3) 1 μL Lipofectamine 2000 and 10-20 ng copGFP expression plasmid were added to Opti-MEM to make the total volume 12.5 μL.
(4) (2) and (3) were mixed to a total volume of 25 μL and incubated for 5-15 minutes, then added to the cell solution in a 48-well plate, placed in a 37 °C incubator for 48-72 hours.

### 3. Observation of Fluorescence of the Reporter System

Human HEK293T cells cultured in a 37 °C incubator for 48 hours were observed under a fluorescence microscope or confocal laser microscope. The efficiency of the prime editor was compared based on the fluorescence, and the fluorescence images were saved.

### 4. Human HEK293T Cell Lysis and Amplicon Sequencing Analysis

### 4.1 Lysis of Human HEK293T Cells

(1) After culturing the cells in a 37 °C incubator for 72 hours, an adjustable-width multi-channel pipette was used to carefully aspirate out the cell culture medium.
(2) Gently 300 μL PBS buffer was added, shaken gently to wash away dead cells, and then the PBS buffer was removed.
(3) 200 μL lysis buffer (proteinase K has been added) was added and treated at 55 °C for 30 minutes.
(4) 50 μL of cell lysis solution was pipetted into a 96-well plate, treated at 95 °C for 5 minutes for later use.

### 4.2 Amplicon Miseq Sequencing Analysis

(1) Miseq one-round primers were used to perform PCR amplification of human HEK293T cell lysate.

The first round of 15 μL amplification system: 7.5 μL 2× Phanta Max Master Mix, 4.5 μL ddH2O, 1 μL Forward primer (10 μM), 1 μL Reverse primer (10 μM), 1 μL cell lysate.

The first round of 15 μL amplification conditions: pre-denaturation at 95 °C for 3 min; denaturation at 95 °C for 15 s, annealing at 50-60 °C for 15 s, extension at 72 °C for 20 s, 34 cycles; full extension at 72 °C for 5 min.
(2) The first-round amplification product was diluted 5 times, and 1 μL was used as the second-round PCR amplification template. The amplification primers were Miseq second-round sequencing primers containing Barcode.

The second round of 25 μL amplification system: 12.5 μL 2× Phanta Max Master Mix, 9.5 uL ddH2O, 1 μL Forward primer (10 μM), 1 μL Reverse primer (10 μM), 1 μL DNA template.

Second-round amplification conditions: pre-denaturation at 95 °C for 3 minutes; denaturation at 95 °C for 15 s, annealing at 50-60 °C for 15 s, extension at 72 °C for 20 s, 10 cycles; full extension at 72 °C for 5 min.
(3) The PCR products were detected by 2% agarose gel electrophoresis, and the target fragments were gel recovered using the AxyPrep DNA Gel Extraction kit. The recovered products were quantitatively analyzed using a NanoDrop ultra-micro spectrophotometer; 100 ng of the recovered products were taken and mixed, and sent to Beijing Qihe Biotechnology Co., Ltd. for amplicon sequencing analysis.
(4) After sequencing was completed, the editing type and editing efficiency of the products at different gene targeting sites in three repeated experiment were compared and analyzed.

### Example 1. The Circular RNA-Dependent Prime Editing System Cas12a-CPE Repaired copGFP to Produce Green Fluorescence in Human 293T Cells

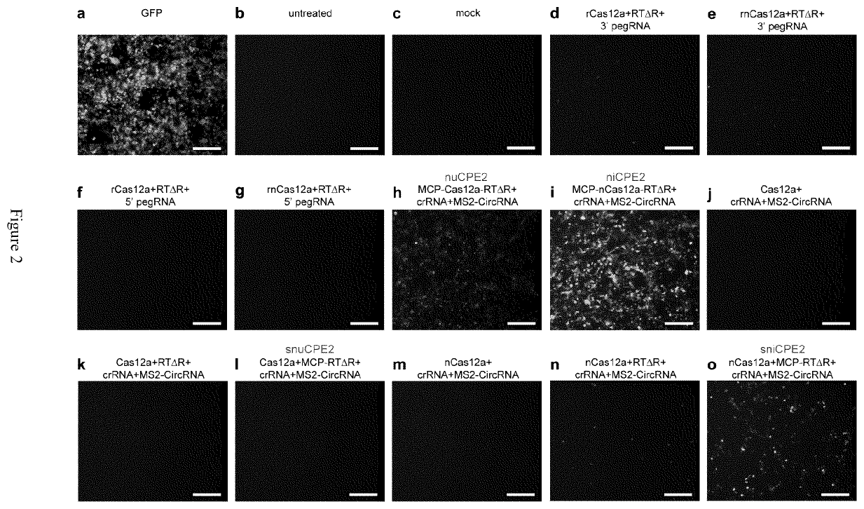

When the inventors previously used pegRNA to develop the Cas12a-PE guided editor, it was found that the efficiency of prime editing was low (FIG. 2). It is believed that because Cas12a itself has the activity of cutting RNA, even if its RNA-cutting domain is mutated, it will still affect pegRNA. Furthermore, because crRNA is very small, only about 20 bp, the RTT and PBS sequences of its pegRNA are very easy to be complementary to the target sequence of crRNA, which greatly hinders the function of pegRNA.

In 2019, Litke and Jaffrey reported the Tornado (Twister-optimized RNA for durable overexpression) human cell circular RNA expression system (https://doi.org/10.1038/s41587-019-0090-6). Tornado uses two ribozymes to cut out the ends of RNA and ligate them into circular RNA under the action of human cell RNA ligase RtcB. In 2022, Sontheimer (https://doi.org/10.1038/s41587-022-01255-9) and Ma Hanhui (https://doi.org/10.1093/procel/pwac014) groups introduced the RTT (reverse transcriptase template) and PBS (primer binding site) sequences of pegRNA (prime editing guide RNA) in the prime editor into circular RNA for expression to generate sPE (split prime editor) prime editor.

The inventors surprisingly found that placing the RTT and PBS sequences of pegRNA in circular RNA for stable expression can well avoid the above two problems of the Cas12a prime editing system.

A prime editor mainly consists of three parts: nCas protein, reverse transcriptase and pegRNA (prime editing guide RNA). When developing the new prime editor Cas12a-CPE, the viral M-MLV reverse transcriptase has higher activity and does not need to be completely changed; while LbCas12a, that is, LbCpf1, does not have the classic nickase form. The inventors found that LbCpf1 with R1138A substitution is the nickase form of LbCpf1 and has nickase activity, and used LbCpf1 with R1138A to construct Cas12a-CPE. The third part of the prime editor is pegRNA. The inventors separated the PBS (primer binding site) and RT (reverse transcriptase) template sequences from the pegRNA and placed them in a circular RNA for stable expression. The inventors also designed a copGFP reporter system, in which the GFP coding sequence contains a three bases mutation. Only when the primer editor achieves precise gene editing can the fluorescence of copGFP be restored.

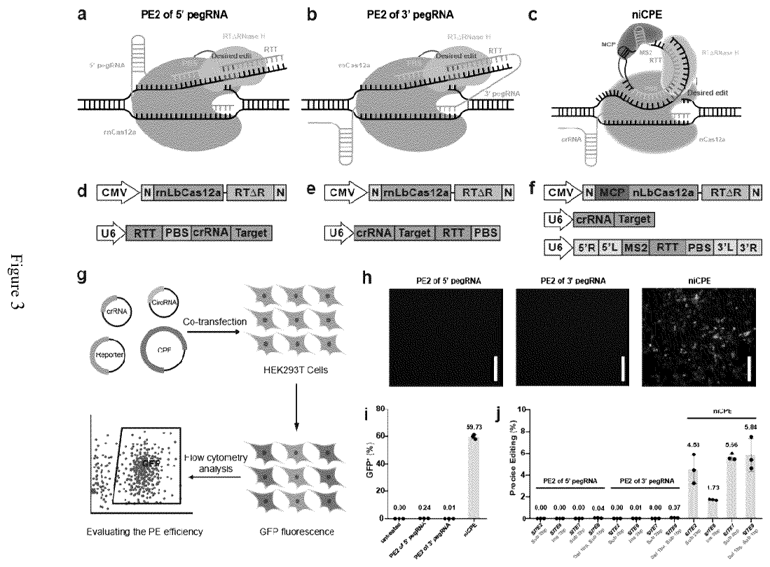

Using the above-mentioned copGFP reporter system, Cas12a-niCPE, Cas12a-nuCPE, Cas12a-sniCPE, Cas12a-snuCPE prime editing system and mutated copGFP plasmid were co-transfected into human HEK293T cells. After approximately 48 hours of culture, significant copGFP fluorescence was detected in the Cas12a-niCPE (FIG. 2i), Cas12a-nuCPE (FIG. 2h) and Cas12a-sniCPE (FIG. 20) reporter systems, and the fluorescence of Cas12a-snuCPE (FIG. 2l) prime editing system is weak. FIG. 2a shows the positive control with copGFP added, the untreated negative control (FIG. 2b) and the control group with only mutated copGFP system (FIG. 2c) had no fluorescence; the Cas12a-prime editing system constructed using 3′pegRNA (FIG. 2d-e) and 5′pegRNA (FIG. 2f-g) had almost no fluorescence. The prime editing system without reverse transcriptase (FIG. 3j, m) had no fluorescence either, and the prime editing system with free reverse transcriptase (FIG. 3k,n) expressed weak fluorescence.

The human HEK293T cell line was further used to test the prime editing reporter systems and the editing efficiency on various targets. It was found that the Cas12a prime editing system constructed using 5′pegRNA and 3′pegRNA had extremely low editing efficiency, while the prime editing system constructed using circular RNA produced precise gene editing (FIG. 3).

### Example 2. The Circular RNA-Dependent Prime Editing System Cas12a-niCPE Achieved Precise Base Editing at the Target Sites in Human 293T Cells

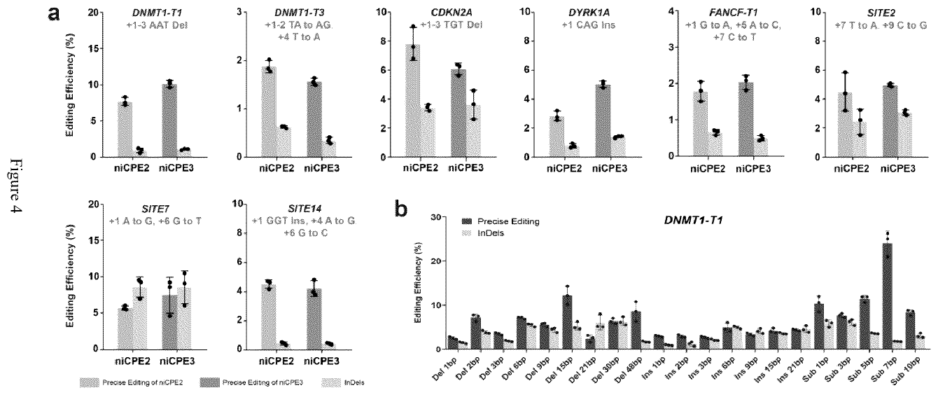

Using the reporter system, the inventors found that the Cas12a-niCPE prime editing system based on circular RNA and non-targeting strand single nickase nCas12a has strong gene editing efficiency, causing the copGFP reporter system to emit strong green fluorescence. The inventors further verified the prime editing efficiency of Cas12a-niCPE in the DNMT1-1 target in human 293T cells and found that there was high prime editing efficiency in base deletion (DEL, deletion), insertion (INS, insertion) and substitution (SUB, substitution), the highest prime editing efficiency reached 24.06% (FIG. 4).

### Example 3. The Circular RNA-Dependent Prime Editing System Cas12a-nuCPE Achieved Precise Base Editing at the Target Sites in Human 293T Cells

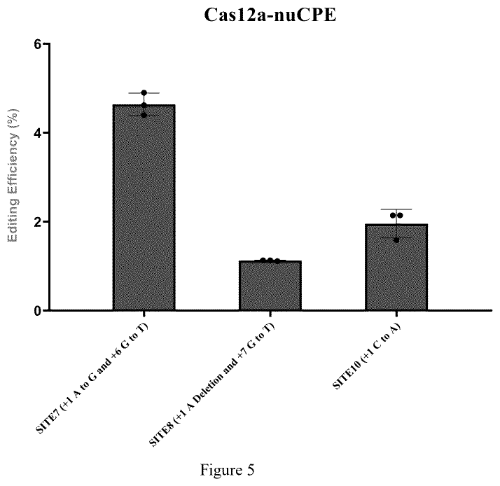

Although the inventors found the non-targeting strand single nickase form of Cas12a (the strand that binds to crRNA is called the targeting strand, and the strand that is complementary to the targeting strand is called the non-targeting strand) Cas12a-R1138A, those newly discovered nuclease may not yet have a non-targeting strand single nickase form, which greatly limits the development of primer editing systems using Cas12b (i.e. C2c1), Cas12c, Cas12d (i.e. CasY), Cas12e (i.e. CasX), Cas12f (i.e. Cas14), Cas12h, Cas12i, Cas12j (i.e. CasΦ), Cas12k, Cas12m, IscB and TnpB and other proteins. Therefore, the inventors first developed the Cas12a-nuCPE prime editing system based on circular RNA using the wild type nuclease Cas12a as a breakthrough. The inventors selected three gene targets in human 293T cells, namely: SITE7, SITE8 and SITE10, to further verify the prime editing efficiency of the Cas12a-nuCPE prime editing system in human cells. After testing, it was found that the Cas12a-nuCPE editing system has prime editing efficiency on all three targets (FIG. 5).

### Example 4. The Detachable Circular RNA-Dependent Prime Editing System Cas12a-sniCPE Achieved Precise Base Editing at Target Sites in Human 293T Cells

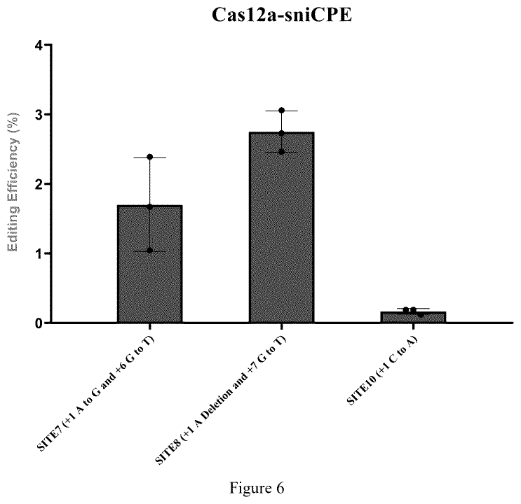

The base editing system has gradually shown surprising prospects in the treatment of human diseases. However, due to the large size of the prime editing system, it is not convenient for the delivery with viral systems such as AAV. Therefore, the inventors further split the MCP-MMLV protein out for expression independent to the expression of nCas12a non-targeting strand single nickase, a splitable prime editing system Cas12a-sniCPE that utilizes non-targeting strand single nickase nCas12a and relies on circular RNA was developed. The three gene targets SITE7, SITE8 and SITE10 in human 293T cells were used to verify the gene editing efficiency of Cas12a-sniCPE, and it was found that the Cas12a-sniCPE editing system had prime editing efficiency on all three targets (FIG. 6).

### Example 5. The Splitable Circular RNA-Dependent Prime Editing System Cas12a-snuCPE Achieved Precise Base Editing at Target Sites in Human 293T Cells

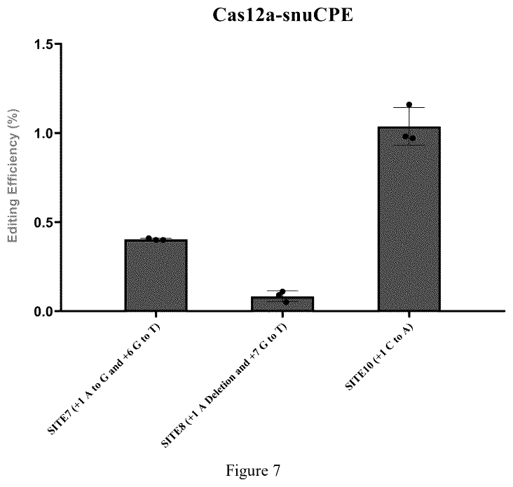

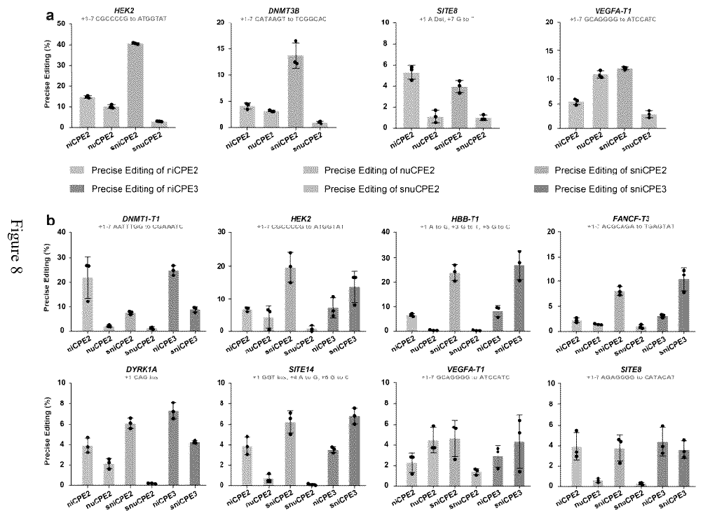

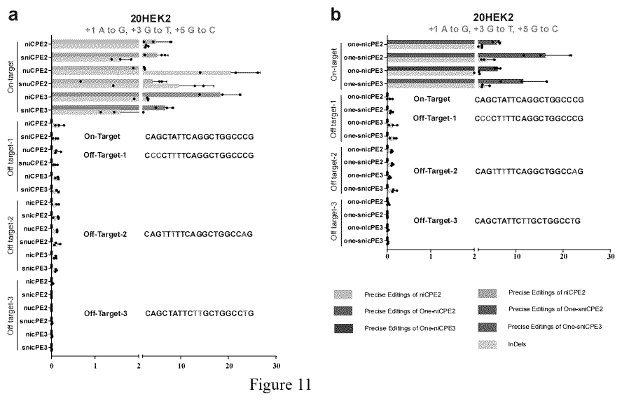

Because the newly discovered nucleases may not yet have a non-targeting strand single nickase form, this greatly limits the development of prime editing systems using proteins such as Cas12b (ie C2c1), Cas12c, Cas12d (ie CasY), Cas12e (ie CasX), Cas12f (ie Cas14) Cas12h, Cas12i, Cas12j (i.e. CasΦ), Cas12k, Cas12m, IscB and TnpB. Therefore, the inventors further developed a splitable prime editing system Cas12a-snuCPE that utilizes nucleases and relies on circular RNA. The three gene targets SITE7, SITE8 and SITE10 in human 293T cells were used to verify the gene editing efficiency of Cas12a-snuCPE, and it was found that the Cas12a-snuCPE editing system had prime editing efficiency on all three targets (FIG. 7). Further research and comparison found that among the niCPE, nuCPE, sniCPE and snuCPE systems, both the niCPE and sniCPE prime editing systems showed better editing efficiency (FIG. 8). At the same time, verification of off-target sites found that the niCPE and sniCPE prime editing systems did not show off-target efficiency in HEK293T (FIG. 11a).

### Example 6. One-CPE Prime Editing System in which all RNAs are Simultaneously Expressed

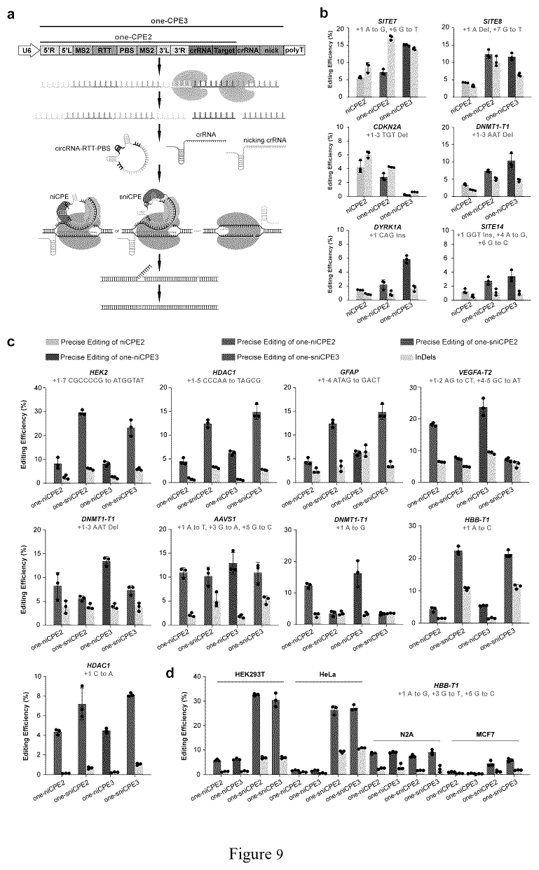

Since Cas12a itself has the function of cutting RNA, a same human U6 promoter can be used to drive the simultaneous expression of multiple tandem RNAs, including crRNA, circular RNA containing RTT and PBS, and nicking RNA. Multiple RNAs are expressed simultaneously under the action of Cas12a; crRNA is used to bind Cas12a protein to find the target site; circular RNA expresses RTT and PBS to provide the starting primer and template for reverse transcriptase. In CPE3, the binding of nicking RNA and Cas12a creates a nick on the distal target strand, further increasing the efficiency of prime editing (FIG. 9a). Further verification in the HEK293T cell line found that one-CPE has efficient prime editing efficiency (FIG. 9b). Verification of off-target sites found that the one-niCPE and one-sniCPE prime editing systems showed no off-target efficiency in HEK293T (FIG. 11b).

### Example 7. Human HEK293T Multigene Cas12a-CPE Prime Editing System

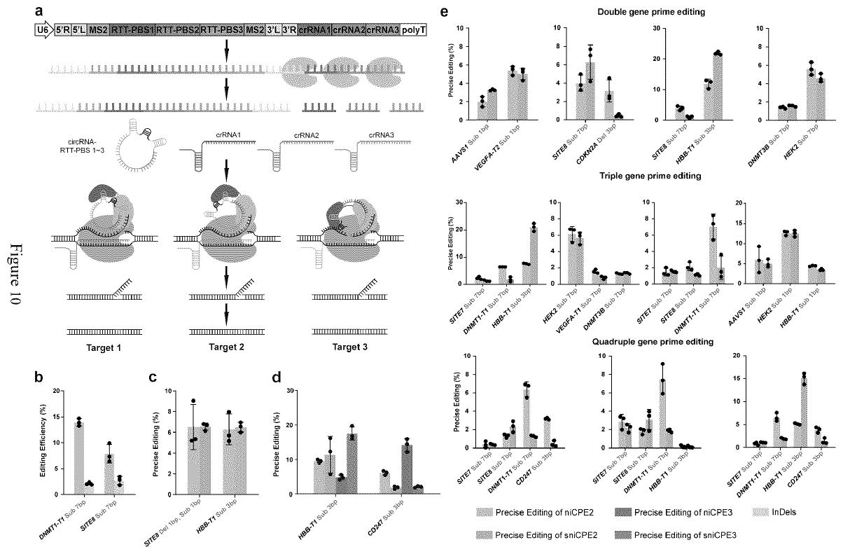

Cas12a protein has been widely used for multiple gene editing in organisms, but it has not yet been used for prime editing. We took advantage of Cas12a's inherent ability to cleave RNA to express multiple crRNAs at the same time. More importantly, we simultaneously included multiple RTT and PBS sequences in tandem in the circular RNA (FIG. 10a). Experiments in the HEK293T human cell line demonstrated that multiple RTTs and PBS expressed in tandem in circular RNAs produced efficient prime editing of different editing sites (FIG. 10b-d). Since many diseases are caused by mutations in multiple targets, we further tested the Cas12a-CPE prime editing system for simultaneous prime editing of two genes, three genes, or even four genes. The results showed that the Cas12a-CPE prime editing system can perform efficient multi-gene prime editing in human HEK293T cells (FIG. 10e).

### Example 8. The Circular RNA-Dependent Prime Editing System Cas12i-niCPE Achieved Precise Base Editing at the Target Sites in Human 293T Cells

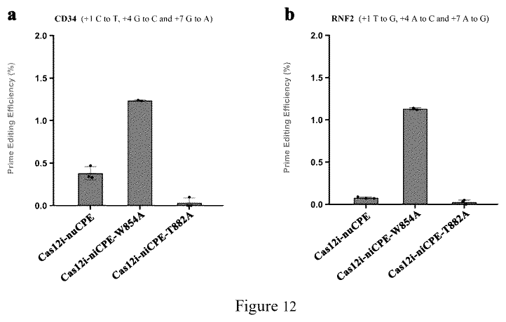

The four prime editing systems developed above based on Cas12a, niCPE, nuCPE, sniCPE, and snuCPE, all have good prime editing efficiency. In particular, the gene editing efficiency of the Cas12a-niCPE prime editing system is already very high. Therefore, the inventor speculates that in most proteins of the newly developed Cas12b (i.e. C2c1), Cas12c, Cas12d (i.e. CasY), Cas12e (i.e. CasX), Cas12f (i.e. Cas14), Cas12h, Cas12i, Cas12j (i.e. CasΦ), Cas12k, Cas12m, IsrB, IscB and TnpB can be applied to the CPE prime editing system. The inventors next selected the Cas1212 protein for development of further CPE-prime editing systems. The inventors first found that two amino acid mutations, W954A and T882A, may cause Cas1212 to be in a non-targeting strand single nickase form. The inventors developed Cas12i-nuCPE, Cas12i-niCPE-W854A, and Cas12i-niCPE-T882A prime editing systems using the wild type Cas1212 and the non-targeting chain single nickase forms obtained by point mutations of these two amino acids. Through the verification of CD34 (FIG. 12a) and RNF2 (FIG. 12b) targets in human 293T cells, it was found that Cas12i-niCPE-W854A had better prime editing efficiency, followed by Cas12i-nuCPE, while Cas12i-niCPE-T882A was less efficient. It shows that W854A is a better non-targeted strand nickase form of Cas12i, and Cas12i-niCPE-W854A will be named Cas 12i-niCPE hereinafter.

Sequences:

SEQ ID NO: 1 LbCas12a amino acid sequence
MSKLEKFTNCYSLSKTLRFKAIPVGKTQENIDNKRLLVEDEKRAEDYKGVKKLLDRYYLSFI
NDVLHSIKLKNLNNYISLFRKKTRTEKENKELENLEINLRKEIAKAFKGNEGYKSLFKKDII
ETILPEFLDDKDEIALVNSFNGFTTAFTGFFDNRENMFSEEAKSTSIAFRCINENLTRYISN
MDIFEKVDAIFDKHEVQEIKEKILNSDYDVEDFFEGEFFNFVLTQEGIDVYNAIIGGFVTES
GEKIKGLNEYINLYNQKTKQKLPKFKPLYKQVLSDRESLSFYGEGYTSDEEVLEVFRNTLNK
NSEIFSSIKKLEKLFKNFDEYSSAGIFVKNGPAISTISKDIFGEWNVIRDKWNAEYDDIHLK
KKAVVTEKYEDDRRKSFKKIGSFSLEQLQEYADADLSVVEKLKEIIIQKVDEIYKVYGSSEK
LFDADFVLEKSLKKNDAVVAIMKDLLDSVKSFENYIKAFFGEGKETNRDESFYGDFVLAYDI
LLKVDHIYDAIRNYVTQKPYSKDKFKLYFQNPQFMGGWDKDKETDYRATILRYGSKYYLAIM
DKKYAKCLQKIDKDDVNGNYEKINYKLLPGPNKMLPKVFFSKKWMAYYNPSEDIQKIYKNGT
FKKGDMFNLNDCHKLIDFFKDSISRYPKWSNAYDFNFSETEKYKDIAGFYREVEEQGYKVSF
ESASKKEVDKLVEEGKLYMFQIYNKDFSDKSHGTPNLHTMYFKLLFDENNHGQIRLSGGAEL
FMRRASLKKEELVVHPANSPIANKNPDNPKKTTTLSYDVYKDKRFSEDQYELHIPIAINKCP
KNIFKINTEVRVLLKHDDNPYVIGIDRGERNLLYIVVVDGKGNIVEQYSLNEIINNFNGIRI
KTDYHSLLDKKEKERFEARQNWTSIENIKELKAGYISQVVHKICELVEKYDAVIALEDLNSG
FKNSRVKVEKQVYQKFEKMLIDKLNYMVDKKSNPCATGGALKGYQITNKFESFKSMSTQNGF
IFYIPAWLTSKIDPSTGFVNLLKTKYTSIADSKKFISSFDRIMYVPEEDLFEFALDYKNFSR
TDADYIKKWKLYSYGNRIRIFRNPKKNNVFDWEEVCLTSAYKELFNKYGINYQQGDIRALLC
EQSDKAFYSSFMALMSLMLQMRNSITGRTDVDFLISPVKNSDGIFYDSRNYEAQENAILPKN
ADANGAYNIARKVLWAIGQFKKAEDEKLDKVKIAISNKEWLEYAQTSVK
SEQ ID NO: 2 LbCas12a(D156R) amino acid sequence
MSKLEKFTNCYSLSKTLRFKAIPVGKTQENIDNKRLLVEDEKRAEDYKGVKKLLDRYYLSFI
NDVLHSIKLKNLNNYISLFRKKTRTEKENKELENLEINLRKEIAKAFKGNEGYKSLFKKDII
ETILPEFLDDKDEIALVNSFNGFTTAFTGFFRNRENMFSEEAKSTSIAFRCINENLTRYISN
MDIFEKVDAIFDKHEVQEIKEKILNSDYDVEDFFEGEFFNFVLTQEGIDVYNAIIGGFVTES
GEKIKGLNEYINLYNQKTKQKLPKFKPLYKQVLSDRESLSFYGEGYTSDEEVLEVFRNTLNK
NSEIFSSIKKLEKLFKNFDEYSSAGIFVKNGPAISTISKDIFGEWNVIRDKWNAEYDDIHLK
KKAVVTEKYEDDRRKSFKKIGSFSLEQLQEYADADLSVVEKLKEIIIQKVDEIYKVYGSSEK
LFDADFVLEKSLKKNDAVVAIMKDLLDSVKSFENYIKAFFGEGKETNRDESFYGDFVLAYDI
LLKVDHIYDAIRNYVTQKPYSKDKFKLYFQNPQFMGGWDKDKETDYRATILRYGSKYYLAIM
DKKYAKCLQKIDKDDVNGNYEKINYKLLPGPNKMLPKVFFSKKWMAYYNPSEDIQKIYKNGT
FKKGDMFNLNDCHKLIDFFKDSISRYPKWSNAYDFNFSETEKYKDIAGFYREVEEQGYKVSF
ESASKKEVDKLVEEGKLYMFQIYNKDFSDKSHGTPNLHTMYFKLLFDENNHGQIRLSGGAEL
FMRRASLKKEELVVHPANSPIANKNPDNPKKTTTLSYDVYKDKRFSEDQYELHIPIAINKCP
KNIFKINTEVRVLLKHDDNPYVIGIDRGERNLLYIVVVDGKGNIVEQYSLNEIINNFNGIRI
KTDYHSLLDKKEKERFEARQNWTSIENIKELKAGYISQVVHKICELVEKYDAVIALEDLNSG
FKNSRVKVEKQVYQKFEKMLIDKLNYMVDKKSNPCATGGALKGYQITNKFESFKSMSTQNGF
IFYIPAWLTSKIDPSTGFVNLLKTKYTSIADSKKFISSFDRIMYVPEEDLFEFALDYKNFSR
TDADYIKKWKLYSYGNRIRIFRNPKKNNVFDWEEVCLTSAYKELFNKYGINYQQGDIRALLC
EQSDKAFYSSFMALMSLMLQMRNSITGRTDVDFLISPVKNSDGIFYDSRNYEAQENAILPKN
ADANGAYNIARKVLWAIGQFKKAEDEKLDKVKIAISNKEWLEYAQTSVK
SEQ ID NO: 3 LbCas12a(D156R, R1138A) amino acid sequence
MSKLEKFTNCYSLSKTLRFKAIPVGKTQENIDNKRLLVEDEKRAEDYKGVKKLLDRYYLSFI
NDVLHSIKLKNLNNYISLFRKKTRTEKENKELENLEINLRKEIAKAFKGNEGYKSLFKKDII
ETILPEFLDDKDEIALVNSFNGFTTAFTGFFRNRENMFSEEAKSTSIAFRCINENLTRYISN
MDIFEKVDAIFDKHEVQEIKEKILNSDYDVEDFFEGEFFNFVLTQEGIDVYNAIIGGFVTES
GEKIKGLNEYINLYNQKTKQKLPKFKPLYKQVLSDRESLSFYGEGYTSDEEVLEVFRNTLNK
NSEIFSSIKKLEKLFKNFDEYSSAGIFVKNGPAISTISKDIFGEWNVIRDKWNAEYDDIHLK
KKAVVTEKYEDDRRKSFKKIGSFSLEQLQEYADADLSVVEKLKEIIIQKVDEIYKVYGSSEK
LFDADFVLEKSLKKNDAVVAIMKDLLDSVKSFENYIKAFFGEGKETNRDESFYGDFVLAYDI
LLKVDHIYDAIRNYVTQKPYSKDKFKLYFQNPQFMGGWDKDKETDYRATILRYGSKYYLAIM
DKKYAKCLQKIDKDDVNGNYEKINYKLLPGPNKMLPKVFFSKKWMAYYNPSEDIQKIYKNGT
FKKGDMFNLNDCHKLIDFFKDSISRYPKWSNAYDFNFSETEKYKDIAGFYREVEEQGYKVSF
ESASKKEVDKLVEEGKLYMFQIYNKDFSDKSHGTPNLHTMYFKLLFDENNHGQIRLSGGAEL
FMRRASLKKEELVVHPANSPIANKNPDNPKKTTTLSYDVYKDKRFSEDQYELHIPIAINKCP
KNIFKINTEVRVLLKHDDNPYVIGIDRGERNLLYIVVVDGKGNIVEQYSLNEIINNFNGIRI
KTDYHSLLDKKEKERFEARQNWTSIENIKELKAGYISQVVHKICELVEKYDAVIALEDLNSG
FKNSRVKVEKQVYQKFEKMLIDKLNYMVDKKSNPCATGGALKGYQITNKFESFKSMSTQNGF
IFYIPAWLTSKIDPSTGFVNLLKTKYTSIADSKKFISSFDRIMYVPEEDLFEFALDYKNFSR
TDADYIKKWKLYSYGNRIRIFRNPKKNNVFDWEEVCLTSAYKELFNKYGINYQQGDIRALLC
EQSDKAFYSSFMALMSLMLQMANSITGRTDVDFLISPVKNSDGIFYDSRNYEAQENAILPKN
ADANGAYNIARKVLWAIGQFKKAEDEKLDKVKIAISNKEWLEYAQTSVK
SEQ ID NO: 4 Cas12i amino acid sequence
MSSAIKSYKSVLRPNERKNQLLKSTIQCLEDGSAFFFKMLQGLFGGITPEIVRFSTEQEKQQ
QDIALWCAVNWFRPVSQDSLTHTIASDNLVEKFEEYYGGTASDAIKQYFSASIGESYYWNDC
RQQYYDLCRELGVEVSDLTHDLEILCREKCLAVATESNQYNSIISVLFGTGRKEDRSVKLRI
TKKILEAISNLKEIPKNVAPIQEIILNVAKATKETFRQVYAGNLGAPSTLERFIAKDGQKEF
DLKKLQTDLKKVIRGKSKERDWCCQEELRSYVEQNTIQYDLWAWGEMFNKAHTALKIKSTRN
YNFAKQRLEQFKRIQSLNNLLVVKKLNDFFDSEFFSGEETYTICVHHLGGKRLSKLYKAWED
DPADPENAIVVLCDDLKNNFKKEPIRNILRYIFTIRQECSAQDILAAAKYNQQLDRYKSQKA
NPSVLGNQGFTWRNAVILPEKAQRNDRPNSLDLRIWLYLKLRHPDGRWKKHHIPFYDTRFFQ
EIYAAGNSPVDTCQFRTPRFGYHLPKLTDQTAIRVNKKHVKAAKTEARIRLAIQQGTLPVSN
LKITRISATINSKGQVRIPVKFDVGRQKGTLQIGDRFCGYDQNQTASHAYSLWEVVKEGQYH
KELGCFVRFISSGDIVSITENRGNQFDQLSYEGLAYPQYADWRKKASKFVSLWQITKKNKKK
EIVTVEAKEKFDAICKYQPRLYKFNKEYAYLLRDIVRGKSLVELQQIRQEIFRFIEQDCGVT
RLGSLSLSTLETVKAVKGIIYSYFSTALNASKNNPISDEQRKEFDPELFALLEKLELIRTRK
KKQKVERIANSLIQTCLENNIKFIRGEGDLSTTNNATKKKANSRSMDWLARGVFNKIRQLAP
MHNITLFGCGSLYTSHQDPLVHRNPDKAMKCRWAAIPVKDIGDWVLRKLSQNLRAKNIGTGE
YYHQGVKEFLSHYELQDLEEELLKWRSDRKSNIPCWVLQNRLAEKLGNKEAVVYIPVRGGRI
YFATHKVATGAVSIVFDQKQVWVCNADHVAAANIALTVKGIGEQSSDEENPDGSRIKLQLTS
SEQ ID NO: 5
Cas12i(W854A) amino acid sequence
MSSAIKSYKSVLRPNERKNQLLKSTIQCLEDGSAFFFKMLQGLFGGITPEIVRFSTEQEKQQ
QDIALWCAVNWFRPVSQDSLTHTIASDNLVEKFEEYYGGTASDAIKQYFSASIGESYYWNDC
RQQYYDLCRELGVEVSDLTHDLEILCREKCLAVATESNQYNSIISVLFGTGRKEDRSVKLRI
TKKILEAISNLKEIPKNVAPIQEIILNVAKATKETFRQVYAGNLGAPSTLERFIAKDGQKEF
DLKKLQTDLKKVIRGKSKERDWCCQEELRSYVEQNTIQYDLWAWGEMFNKAHTALKIKSTRN
YNFAKQRLEQFKRIQSLNNLLVVKKLNDFFDSEFFSGEETYTICVHHLGGKRLSKLYKAWED
DPADPENAIVVLCDDLKNNFKKEPIRNILRYIFTIRQECSAQDILAAAKYNQQLDRYKSQKA
NPSVLGNQGFTWRNAVILPEKAQRNDRPNSLDLRIWLYLKLRHPDGRWKKHHIPFYDTRFFQ
EIYAAGNSPVDTCQFRTPRFGYHLPKLTDQTAIRVNKKHVKAAKTEARIRLAIQQGTLPVSN
LKITRISATINSKGQVRIPVKFDVGRQKGTLQIGDRFCGYDQNQTASHAYSLWEVVKEGQYH
KELGCFVRFISSGDIVSITENRGNQFDQLSYEGLAYPQYADWRKKASKFVSLWQITKKNKKK
EIVTVEAKEKFDAICKYQPRLYKFNKEYAYLLRDIVRGKSLVELQQIRQEIFRFIEQDCGVT
RLGSLSLSTLETVKAVKGIIYSYFSTALNASKNNPISDEQRKEFDPELFALLEKLELIRTRK
KKQKVERIANSLIQTCLENNIKFIRGEGDLSTTNNATKKKANSRSMDALARGVFNKIRQLAP
MHNITLFGCGSLYTSHQDPLVHRNPDKAMKCRWAAIPVKDIGDWVLRKLSQNLRAKNIGTGE
YYHQGVKEFLSHYELQDLEEELLKWRSDRKSNIPCWVLQNRLAEKLGNKEAVVYIPVRGGRI
YFATHKVATGAVSIVFDQKQVWVCNADHVAAANIALTVKGIGEQSSDEENPDGSRIKLQLTS
SEQ ID NO: 6 M-MLV reverse transcriptase amino acid sequence
STLNIEDEYRLHETSKEPDVSLGSTWLSDFPQAWAETGGMGLAVRQAPLIIPLKATSTPVSI
KQYPMSQEARLGIKPHIQRLLDQGILVPCQSPWNTPLLPVKKPGTNDYRPVQDLREVNKRVE
DIHPTVPNPYNLLSGLPPSHQWYTVLDLKDAFFCLRLHPTSQPLFAFEWRDPEMGISGQLTW
TRLPQGFKNSPTLFNEALHRDLADFRIQHPDLILLQYVDDLLLAATSELDCQQGTRALLQTL
GNLGYRASAKKAQICQKQVKYLGYLLKEGQRWLTEARKETVMGQPTPKTPRQLREFLGKAGF
CRLFIPGFAEMAAPLYPLTKPGTLFNWGPDQQKAYQEIKQALLTAPALGLPDLTKPFELFVD
EKQGYAKGVLTQKLGPWRRPVAYLSKKLDPVAAGWPPCLRMVAAIAVLTKDAGKLTMGQPLV
ILAPHAVEALVKQPPDRWLSNARMTHYQALLLDTDRVQFGPVVALNPATLLPLPEEGLQHNC
LDILAEAHGTRPDLTDQPLPDADHTWYTDGSSLLQEGQRKAGAAVTTETEVIWAKALPAGTS
AQRAELIALTQALKMAEGKKLNVYTDSRYAFATAHIHGEIYRRRGWLTSEGKEIKNKDEILA
LLKALFLPKRLSIIHCPGHQKGHSAEARGNRMADQAARKAAITETPDTSTLLIENSSP
SEQ ID NO: 7 M-MLV reverse transcriptase (ΔRnaseH) amino acid sequence
TLNIEDEYRLHETSKEPDVSLGSTWLSDFPQAWAETGGMGLAVRQAPLIIPLKATSTPVSIK
QYPMSQEARLGIKPHIQRLLDQGILVPCQSPWNTPLLPVKKPGTNDYRPVQDLREVNKRVED
IHPTVPNPYNLLSGLPPSHQWYTVLDLKDAFFCLRLHPTSQPLFAFEWRDPEMGISGQLTWT
RLPQGFKNSPTLFNEALHRDLADFRIQHPDLILLQYVDDLLLAATSELDCQQGTRALLQTLG
NLGYRASAKKAQICQKQVKYLGYLLKEGQRWLTEARKETVMGQPTPKTPRQLREFLGKAGFC
RLFIPGFAEMAAPLYPLTKPGTLFNWGPDQQKAYQEIKQALLTAPALGLPDLTKPFELFVDE
KQGYAKGVLTQKLGPWRRPVAYLSKKLDPVAAGWPPCLRMVAAIAVLTKDAGKLTMGQPLVI
LAPHAVEALVKQPPDRWLSNARMTHYQALLLDTDRVQFGPVVALNPATLLPLPEEGLQHNCL
DILAEAHGTRPDLTDQPL
SEQ ID NO: 8 MCP amino acid sequence
ASNFTQFVLVDNGGTGDVTVAPSNFANGIAEWISSNSRSQAYKVTCSVRQSSAQNRKYTIKV
EVPKGAWRSYLNMELTIPIFATNSDCELIVKAMQGLLKDGNPIPSAIAANSGIY
SEQ ID NO: 9 32 aa linker amino acid sequence
SGGSSGGSSGSETPGTSESATPESSGGSSGGS
SEQ ID NO: 10 NLS amino acid sequence
MKRTADGSEFESPKKKRKV
SEQ ID NO: 11 NLS amino acid sequence
KRTADGSEFEPKKKRKV
SEQ ID NO: 12 Cas12a-niCPE: NLS-MCP-32 aa Linker-nCas12a(D156R, R1138A)-MMLV
ΔRnaseH-NLS
MKRTADGSEFESPKKKRKVKLASNFTQFVLVDNGGTGDVTVAPSNFANGIAEWISSNSRSQA
YKVTCSVRQSSAQNRKYTIKVEVPKGAWRSYLNMELTIPIFATNSDCELIVKAMQGLLKDGN
PIPSAIAANSGIYSRLQSGGSSGGSSGSETPGTSESATPESSGGSSGGSELMSKLEKFTNCY
SLSKTLRFKAIPVGKTQENIDNKRLLVEDEKRAEDYKGVKKLLDRYYLSFINDVLHSIKLKN
LNNYISLFRKKTRTEKENKELENLEINLRKEIAKAFKGNEGYKSLFKKDIIETILPEFLDDK
DEIALVNSFNGFTTAFTGFFRNRENMFSEEAKSTSIAFRCINENLTRYISNMDIFEKVDAIF
DKHEVQEIKEKILNSDYDVEDFFEGEFFNFVLTQEGIDVYNAIIGGFVTESGEKIKGLNEYI
NLYNQKTKQKLPKFKPLYKQVLSDRESLSFYGEGYTSDEEVLEVFRNTLNKNSEIFSSIKKL
EKLFKNFDEYSSAGIFVKNGPAISTISKDIFGEWNVIRDKWNAEYDDIHLKKKAVVTEKYED
DRRKSFKKIGSFSLEQLQEYADADLSVVEKLKEIIIQKVDEIYKVYGSSEKLFDADFVLEKS
LKKNDAVVAIMKDLLDSVKSFENYIKAFFGEGKETNRDESFYGDFVLAYDILLKVDHIYDAI
RNYVTQKPYSKDKFKLYFQNPQFMGGWDKDKETDYRATILRYGSKYYLAIMDKKYAKCLQKI
DKDDVNGNYEKINYKLLPGPNKMLPKVFFSKKWMAYYNPSEDIQKIYKNGTFKKGDMFNLND
CHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEE
LVVHPANSPIANKNPDNPKKTTTLSYDVYKDKRFSEDQYELHIPIAINKCPKNIFKINTEVR
VLLKHDDNPYVIGIDRGERNLLYIVVVDGKGNIVEQYSLNEIINNFNGIRIKTDYHSLLDKK
EKERFEARQNWTSIENIKELKAGYISQVVHKICELVEKYDAVIALEDLNSGFKNSRVKVEKQ
VYQKFEKMLIDKLNYMVDKKSNPCATGGALKGYQITNKFESFKSMSTQNGFIFYIPAWLTSK
IDPSTGFVNLLKTKYTSIADSKKFISSFDRIMYVPEEDLFEFALDYKNFSRTDADYIKKWKL
YSYGNRIRIFRNPKKNNVFDWEEVCLTSAYKELFNKYGINYQQGDIRALLCEQSDKAFYSSF
MALMSLMLQMANSITGRTDVDFLISPVKNSDGIFYDSRNYEAQENAILPKNADANGAYNIAR
KVLWAIGQFKKAEDEKLDKVKIAISNKEWLEYAQTSVKSGGSSGGSSGSETPGTSESATPES
SGGSSGGSPGSTLNIEDEYRLHETSKEPDVSLGSTWLSDFPQAWAETGGMGLAVRQAPLIIP
LKATSTPVSIKQYPMSQEARLGIKPHIQRLLDQGILVPCQSPWNTPLLPVKKPGTNDYRPVQ
DLREVNKRVEDIHPTVPNPYNLLSGLPPSHQWYTVLDLKDAFFCLRLHPTSQPLFAFEWRDP
EMGISGQLTWTRLPQGFKNSPTLFNEALHRDLADFRIQHPDLILLQYVDDLLLAATSELDCQ
QGTRALLQTLGNLGYRASAKKAQICQKQVKYLGYLLKEGQRWLTEARKETVMGQPTPKTPRQ
LREFLGKAGFCRLFIPGFAEMAAPLYPLTKPGTLFNWGPDQQKAYQEIKQALLTAPALGLPD
LTKPFELFVDEKQGYAKGVLTQKLGPWRRPVAYLSKKLDPVAAGWPPCLRMVAAIAVLTKDA
GKLTMGQPLVILAPHAVEALVKQPPDRWLSNARMTHYQALLLDTDRVQFGPVVALNPATLLP
LPEEGLQHNCLDILAEAHGTRPDLTDQPLSGGSKRTADGSEFEPKKKRKV*
SEQ ID NO: 13 Cas12a-nuCPE: NLS-MCP-32aa Linker-Cas12a(D156R)-MMLVΔRnaseH-
NLS
MKRTADGSEFESPKKKRKVKLASNFTQFVLVDNGGTGDVTVAPSNFANGIAEWISSNSRSQA
YKVTCSVRQSSAQNRKYTIKVEVPKGAWRSYLNMELTIPIFATNSDCELIVKAMQGLLKDGN
PIPSAIAANSGIYSRLQSGGSSGGSSGSETPGTSESATPESSGGSSGGSELMSKLEKFTNCY
SLSKTLRFKAIPVGKTQENIDNKRLLVEDEKRAEDYKGVKKLLDRYYLSFINDVLHSIKLKN
LNNYISLFRKKTRTEKENKELENLEINLRKEIAKAFKGNEGYKSLFKKDIIETILPEFLDDK
DEIALVNSFNGFTTAFTGFFRNRENMFSEEAKSTSIAFRCINENLTRYISNMDIFEKVDAIF
DKHEVQEIKEKILNSDYDVEDFFEGEFFNFVLTQEGIDVYNAIIGGFVTESGEKIKGLNEYI
NLYNQKTKQKLPKFKPLYKQVLSDRESLSFYGEGYTSDEEVLEVFRNTLNKNSEIFSSIKKL
EKLFKNFDEYSSAGIFVKNGPAISTISKDIFGEWNVIRDKWNAEYDDIHLKKKAVVTEKYED
DRRKSFKKIGSFSLEQLQEYADADLSVVEKLKEIIIQKVDEIYKVYGSSEKLFDADFVLEKS
LKKNDAVVAIMKDLLDSVKSFENYIKAFFGEGKETNRDESFYGDFVLAYDILLKVDHIYDAI
RNYVTQKPYSKDKFKLYFQNPQFMGGWDKDKETDYRATILRYGSKYYLAIMDKKYAKCLQKI
DKDDVNGNYEKINYKLLPGPNKMLPKVFFSKKWMAYYNPSEDIQKIYKNGTFKKGDMFNLND
CHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEE
LVVHPANSPIANKNPDNPKKTTTLSYDVYKDKRFSEDQYELHIPIAINKCPKNIFKINTEVR
VLLKHDDNPYVIGIDRGERNLLYIVVVDGKGNIVEQYSLNEIINNFNGIRIKTDYHSLLDKK
EKERFEARQNWTSIENIKELKAGYISQVVHKICELVEKYDAVIALEDLNSGFKNSRVKVEKQ
VYQKFEKMLIDKLNYMVDKKSNPCATGGALKGYQITNKFESFKSMSTQNGFIFYIPAWLTSK
IDPSTGFVNLLKTKYTSIADSKKFISSFDRIMYVPEEDLFEFALDYKNFSRTDADYIKKWKL
YSYGNRIRIFRNPKKNNVFDWEEVCLTSAYKELFNKYGINYQQGDIRALLCEQSDKAFYSSF
MALMSLMLQMRNSITGRTDVDFLISPVKNSDGIFYDSRNYEAQENAILPKNADANGAYNIAR
KVLWAIGQFKKAEDEKLDKVKIAISNKEWLEYAQTSVKSGGSSGGSSGSETPGTSESATPES
SGGSSGGSPGSTLNIEDEYRLHETSKEPDVSLGSTWLSDFPQAWAETGGMGLAVRQAPLIIP
LKATSTPVSIKQYPMSQEARLGIKPHIQRLLDQGILVPCQSPWNTPLLPVKKPGTNDYRPVQ
DLREVNKRVEDIHPTVPNPYNLLSGLPPSHQWYTVLDLKDAFFCLRLHPTSQPLFAFEWRDP
EMGISGQLTWTRLPQGFKNSPTLFNEALHRDLADFRIQHPDLILLQYVDDLLLAATSELDCQ
QGTRALLQTLGNLGYRASAKKAQICQKQVKYLGYLLKEGQRWLTEARKETVMGQPTPKTPRQ
LREFLGKAGFCRLFIPGFAEMAAPLYPLTKPGTLFNWGPDQQKAYQEIKQALLTAPALGLPD
LTKPFELFVDEKQGYAKGVLTQKLGPWRRPVAYLSKKLDPVAAGWPPCLRMVAAIAVLTKDA
GKLTMGQPLVILAPHAVEALVKQPPDRWLSNARMTHYQALLLDTDRVQFGPVVALNPATLLP
LPEEGLQHNCLDILAEAHGTRPDLTDQPLSGGSKRTADGSEFEPKKKRKV*
SEQ ID NO: 14 Cas12i-niCPE: NLS-MCP-32 aa Linker-Cas1212max(W854A)-
MMLVΔRnaseH-NLS
MKRTADGSEFESPKKKRKVKLASNFTQFVLVDNGGTGDVTVAPSNFANGIAEWISSNSRSQA
YKVTCSVRQSSAQNRKYTIKVEVPKGAWRSYLNMELTIPIFATNSDCELIVKAMQGLLKDGN
PIPSAIAANSGIYSRLQSGGSSGGSSGSETPGTSESATPESSGGSSGGSELMSSAIKSYKSV
LRPNERKNQLLKSTIQCLEDGSAFFFKMLQGLFGGITPEIVRFSTEQEKQQQDIALWCAVNW
FRPVSQDSLTHTIASDNLVEKFEEYYGGTASDAIKQYFSASIGESYYWNDCRQQYYDLCREL
GVEVSDLTHDLEILCREKCLAVATESNQYNSIISVLFGTGRKEDRSVKLRITKKILEAISNL
KEIPKNVAPIQEIILNVAKATKETFRQVYAGNLGAPSTLERFIAKDGQKEFDLKKLQTDLKK
VIRGKSKERDWCCQEELRSYVEQNTIQYDLWAWGEMFNKAHTALKIKSTRNYNFAKQRLEQF
KRIQSLNNLLVVKKLNDFFDSEFFSGEETYTICVHHLGGKRLSKLYKAWEDDPADPENAIVV
LCDDLKNNFKKEPIRNILRYIFTIRQECSAQDILAAAKYNQQLDRYKSQKANPSVLGNQGFT
WRNAVILPEKAQRNDRPNSLDLRIWLYLKLRHPDGRWKKHHIPFYDTRFFQEIYAAGNSPVD
TCQFRTPRFGYHLPKLTDQTAIRVNKKHVKAAKTEARIRLAIQQGTLPVSNLKITRISATIN
SKGQVRIPVKFDVGRQKGTLQIGDRFCGYDQNQTASHAYSLWEVVKEGQYHKELGCFVRFIS
SGDIVSITENRGNQFDQLSYEGLAYPQYADWRKKASKFVSLWQITKKNKKKEIVTVEAKEKF
DAICKYQPRLYKFNKEYAYLLRDIVRGKSLVELQQIRQEIFRFIEQDCGVTRLGSLSLSTLE
TVKAVKGIIYSYFSTALNASKNNPISDEQRKEFDPELFALLEKLELIRTRKKKQKVERIANS
LIQTCLENNIKFIRGEGDLSTTNNATKKKANSRSMDALARGVFNKIRQLAPMHNITLFGCGS
LYTSHQDPLVHRNPDKAMKCRWAAIPVKDIGDWVLRKLSQNLRAKNIGTGEYYHQGVKEFLS
HYELQDLEEELLKWRSDRKSNIPCWVLQNRLAEKLGNKEAVVYIPVRGGRIYFATHKVATGA
VSIVFDQKQVWVCNADHVAAANIALTVKGIGEQSSDEENPDGSRIKLQLTSSGGSSGGSSGS
ETPGTSESATPESSGGSSGGSPGSTLNIEDEYRLHETSKEPDVSLGSTWLSDFPQAWAETGG
MGLAVRQAPLIIPLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLDLKDAFFCLRLHP
TSQPLFAFEWRDPEMGISGQLTWTRLPQGFKNSPTLFNEALHRDLADFRIQHPDLILLQYVD
DLLLAATSELDCQQGTRALLQTLGNLGYRASAKKAQICQKQVKYLGYLLKEGQRWLTEARKE
TVMGQPTPKTPRQLREFLGKAGFCRLFIPGFAEMAAPLYPLTKPGTLFNWGPDQQKAYQEIK
QALLTAPALGLPDLTKPFELFVDEKQGYAKGVLTQKLGPWRRPVAYLSKKLDPVAAGWPPCL
RMVAAIAVLTKDAGKLTMGQPLVILAPHAVEALVKQPPDRWLSNARMTHYQALLLDTDRVQF
GPVVALNPATLLPLPEEGLQHNCLDILAEAHGTRPDLTDQPLSGGSKRTADGSEFEPKKKRK
V*
SEQ ID NO: 15 Cas12i-nuCPE: NLS-MCP-32 aa Linker-Cas1212max-MMLVΔRnaseH-NLS
MKRTADGSEFESPKKKRKVKLASNFTQFVLVDNGGTGDVTVAPSNFANGIAEWISSNSRSQA
YKVTCSVRQSSAQNRKYTIKVEVPKGAWRSYLNMELTIPIFATNSDCELIVKAMQGLLKDGN
PIPSAIAANSGIYSRLQSGGSSGGSSGSETPGTSESATPESSGGSSGGSELMSSAIKSYKSV
LRPNERKNQLLKSTIQCLEDGSAFFFKMLQGLFGGITPEIVRFSTEQEKQQQDIALWCAVNW
FRPVSQDSLTHTIASDNLVEKFEEYYGGTASDAIKQYFSASIGESYYWNDCRQQYYDLCREL
GVEVSDLTHDLEILCREKCLAVATESNQYNSIISVLFGTGRKEDRSVKLRITKKILEAISNL
KEIPKNVAPIQEIILNVAKATKETFRQVYAGNLGAPSTLERFIAKDGQKEFDLKKLQTDLKK
VIRGKSKERDWCCQEELRSYVEQNTIQYDLWAWGEMFNKAHTALKIKSTRNYNFAKQRLEQF
KRIQSLNNLLVVKKLNDFFDSEFFSGEETYTICVHHLGGKRLSKLYKAWEDDPADPENAIVV
LCDDLKNNFKKEPIRNILRYIFTIRQECSAQDILAAAKYNQQLDRYKSQKANPSVLGNQGFT
WRNAVILPEKAQRNDRPNSLDLRIWLYLKLRHPDGRWKKHHIPFYDTRFFQEIYAAGNSPVD
TCQFRTPRFGYHLPKLTDQTAIRVNKKHVKAAKTEARIRLAIQQGTLPVSNLKITRISATIN
SKGQVRIPVKFDVGRQKGTLQIGDRFCGYDQNQTASHAYSLWEVVKEGQYHKELGCFVRFIS
SGDIVSITENRGNQFDQLSYEGLAYPQYADWRKKASKFVSLWQITKKNKKKEIVTVEAKEKF
DAICKYQPRLYKFNKEYAYLLRDIVRGKSLVELQQIRQEIFRFIEQDCGVTRLGSLSLSTLE
TVKAVKGIIYSYFSTALNASKNNPISDEQRKEFDPELFALLEKLELIRTRKKKQKVERIANS
LIQTCLENNIKFIRGEGDLSTTNNATKKKANSRSMDWLARGVFNKIRQLAPMHNITLFGCGS
LYTSHQDPLVHRNPDKAMKCRWAAIPVKDIGDWVLRKLSQNLRAKNIGTGEYYHQGVKEFLS
HYELQDLEEELLKWRSDRKSNIPCWVLQNRLAEKLGNKEAVVYIPVRGGRIYFATHKVATGA
VSIVFDQKQVWVCNADHVAAANIALTVKGIGEQSSDEENPDGSRIKLQLTSSGGSSGGSSGS
ETPGTSESATPESSGGSSGGSPGSTLNIEDEYRLHETSKEPDVSLGSTWLSDFPQAWAETGG
MGLAVRQAPLIIPLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLDLKDAFFCLRLHP
TSQPLFAFEWRDPEMGISGQLTWTRLPQGFKNSPTLFNEALHRDLADFRIQHPDLILLQYVD
DLLLAATSELDCQQGTRALLQTLGNLGYRASAKKAQICQKQVKYLGYLLKEGQRWLTEARKE
TVMGQPTPKTPRQLREFLGKAGFCRLFIPGFAEMAAPLYPLTKPGTLFNWGPDQQKAYQEIK
QALLTAPALGLPDLTKPFELFVDEKQGYAKGVLTQKLGPWRRPVAYLSKKLDPVAAGWPPCL
RMVAAIAVLTKDAGKLTMGQPLVILAPHAVEALVKQPPDRWLSNARMTHYQALLLDTDRVQF
GPVVALNPATLLPLPEEGLQHNCLDILAEAHGTRPDLTDQPLSGGSKRTADGSEFEPKKKRK
V*
SEQ ID NO: 16 MCP-MMLV: NLS-MCP-MMLV-NLS
MKRTADGSEFESPKKKRKVKLASNFTQFVLVDNGGTGDVTVAPSNFANGIAEWISSNSRSQA
YKVTCSVRQSSAQNRKYTIKVEVPKGAWRSYLNMELTIPIFATNSDCELIVKAMQGLLKDGN
PIPSAIAANSGIYSRSGGSSGGSSGSETPGTSESATPESSGGSSGGSSTLNIEDEYRLHETS
KEPDVSLGSTWLSDFPQAWAETGGMGLAVRQAPLIIPLKATSTPVSIKQYPMSQEARLGIKP
HIQRLLDQGILVPCQSPWNTPLLPVKKPGTNDYRPVQDLREVNKRVEDIHPTVPNPYNLLSG
LPPSHQWYTVLDLKDAFFCLRLHPTSQPLFAFEWRDPEMGISGQLTWTRLPQGFKNSPTLFN
EALHRDLADFRIQHPDLILLQYVDDLLLAATSELDCQQGTRALLQTLGNLGYRASAKKAQIC
QKQVKYLGYLLKEGQRWLTEARKETVMGQPTPKTPRQLREFLGKAGFCRLFIPGFAEMAAPL
YPLTKPGTLFNWGPDQQKAYQEIKQALLTAPALGLPDLTKPFELFVDEKQGYAKGVLTQKLG
PWRRPVAYLSKKLDPVAAGWPPCLRMVAAIAVLTKDAGKLTMGQPLVILAPHAVEALVKQPP
DRWLSNARMTHYQALLLDTDRVQFGPVVALNPATLLPLPEEGLQHNCLDILAEAHGTRPDLT
DQPLPDADHTWYTDGSSLLQEGQRKAGAAVTTETEVIWAKALPAGTSAQRAELIALTQALKM
AEGKKLNVYTDSRYAFATAHIHGEIYRRRGWLTSEGKEIKNKDEILALLKALFLPKRLSIIH
CPGHQKGHSAEARGNRMADQAARKAAITETPDTSTLLIENSSPSGGSKRTADGSEFEPKKKR
KV*
SEQ ID NO: 17 MS2 sequence
GCACATGAGGATCACCCATGTGC
SEQ ID NO: 18 hU6-5′ + 3′MS2-CircularRNA: hU6-Ribozyme1-Arm1-5′MS2-BsaI × 2-
3′MS2-Arm2-Ribozyme2-polyT
GAGGGCCTATTTCCCATGATTCCTTCATATTTGCATATACGATACAAGGCTGTTAGAGAGAT
AATTAGAATTAATTTGACTGTAAACACAAAGATATTAGTACAAAATACGTGACGTAGAAAGT
AATAATTTCTTGGGTAGTTTGCAGTTTTAAAATTATGTTTTAAAATGGACTATCATATGCTT
ACCGTAACTTGAAAGTATTTCGATTTCTTGGCTTTATATATCTTGTGGAAAGGACGAAACAC
CGGCCATCAGTCGCCGGTCCCAAGCCCGGATAAAATGGGAGGGGGGGGGAAACCGCCTAACC
ATGCCGACTGATGGCAGAACTAAGCACATGAGGATCACCCATGTGCCGAGACCTTCCGATAG
GTCTCCAAGGCACATGAGGATCACCCATGTGCTAAATTAACACTGCCATCAGTCGGCGTGGA
CTGTAGAACACTGCCAATGCCGGTCCCAAGCCCGGATAAAAGTGGAGGGTACAGTCCACGCT
TTTTTTT
SEQ ID NO: 19 5′ Ribozyme coding sequence
GCCATCAGTCGCCGGTCCCAAGCCCGGATAAAATGGGAGGGGGCGGGAAACCGCCT
SEQ ID NO: 20 3′ Ribozyme coding sequence
AACACTGCCAATGCCGGTCCCAAGCCCGGATAAAAGTGGAGGGTACAGTCCACGC
SEQ ID NO: 21 5′ circularization arm
AACCATGCCGACTGATGGCAG
SEQ ID NO: 22 3′ circularization arm
CTGCCATCAGTCGGCGTGGACTGTAG
SEQ ID NO: 23 Cas12a-crRNA scaffold coding sequence
TAATTTCTACTAAGTGTAGAT
SEQ ID NO: 24 Cas12i-crRNA scaffold coding sequence
AGAAATCCGTCTTTCATTGACGG

What we claim are:

1. A prime editing system for targeted modification of a genomic DNA sequence of an organism, which comprises:
i) a) a CRISPR nuclease and/or an expression construct containing a nucleotide sequence encoding said CRISPR nuclease, and a reverse transcriptase and/or an expression construct containing a nucleotide sequence encoding said reverse transcriptase, or
b) a prime editing fusion protein and/or an expression construct containing a nucleotide sequence encoding the prime editing fusion protein, wherein the prime editing fusion protein comprises a CRISPR nuclease and a reverse transcriptase;
ii) an editing guide RNA (gRNA) directed to a target sequence in the genomic DNA and/or an expression construct containing a nucleotide sequence encoding said editing gRNA; and
iii) a circular RNA containing a reverse transcription template (RTT) sequence and a primer binding site (PBS) sequence, and/or an expression construct containing a nucleotide sequence encoding said circular RNA,
preferably, the CRISPR nuclease is not a Cas9 nuclease or a variant thereof.

2. The prime editing system of claim 1, wherein the CRISPR nuclease is selected from the group consisting of Cas12a (i.e. Cpf1), Cas12b (i.e. C2c1), Cas12c, Cas12d (i.e. CasY), Cas12e (i.e. CasX), Cas12f (i.e. Cas14), Cas12h, Cas12i, Cas12j (i.e. CasΦ), Cas12k, Cas12m, IsrB, IscB and TnpB.

3. The prime editing system of claim 1 or 2, wherein the CRISPR nuclease is in its nickase form.

4. The prime editing system of claim 1 or 2, wherein the CRISPR nuclease is Cas12a, which comprises an amino acid sequence shown in SEQ ID NO: 1 or 2.

5. The prime editing system of claim 1 or 2, wherein the CRISPR nuclease is a Cas12a nickase, in which the amino acid at position 1138 is substituted relative to SEQ ID NO: 1 or 2, for example, amino acid R at position 1138 is replaced by A, preferably, the CRISPR nuclease comprises the amino acid sequence shown in SEQ ID NO:3.

6. The prime editing system of claim 1 or 2, wherein the CRISPR nuclease is Cas12i, which comprises the amino acid sequence shown in SEQ ID NO:4.

7. The prime editing system of claim 1 or 2, wherein the CRISPR nuclease is a Cas12i nickase, in which the amino acid at position 854 is substituted relative to SEQ ID NO: 4, for example, the amino acid W at position 854 is replaced by A, preferably, the Cas12i nickase comprises the amino acid sequence shown in SEQ ID NO:5.

8. The prime editing system of any one of claims 1-7, wherein the reverse transcriptase is an M-MLV reverse transcriptase, such as an M-MLV reverse transcriptase comprising the amino acid sequence shown in SEQ ID NO: 6.

9. The prime editing system of claim 8, wherein the RNase H domain of the M-MLV reverse transcriptase is mutated or deleted.

10. The prime editing system of claim 9, wherein the M-MLV reverse transcriptase in which the RNase H domain is mutated or deleted comprises the amino acid sequence shown in SEQ ID NO: 7.

11. The prime editing system of any one of claims 1-10, wherein the reverse transcriptase and/or the prime editing fusion protein further comprise one or more RNA aptamer binding protein sequences.

12. The prime editing system of claim 11, wherein the RNA adapter binding protein is an MCP protein, for example, the MCP comprises the amino acid sequence shown in SEQ ID NO: 8.

13. The prime editing system of claim 1, wherein the prime editing fusion protein of i)-b) comprises an amino acid sequence selected from SEQ ID NO: 12-15, preferably comprises the amino acid sequence of SEQ ID NO: 12.

14. The prime editing system of claim 1, wherein the CRISPR nuclease of i)-a) comprises an amino acid sequence selected from SEQ ID NO: 1-5, preferably comprises an amino acid sequence of SEQ ID NO: 3 or 5; the reverse transcriptase comprises the amino acid sequence shown in SEQ ID NO: 16.

15. The prime editing system of any one of claims 1-14, wherein the guide RNA (gRNA) comprises a scaffold sequence shown in SEQ ID NO: 23 or 24.

16. The prime editing system of any one of claims 1-15, wherein in the circular RNA, the reverse transcription template (RTT) sequence and the primer binding site (PBS) sequence are directly linked, and the RTT sequence is located to the 5′ end of the PBS sequence.

17. The prime editing system of any one of claims 1-16, wherein the circular RNA further comprises one or more RNA adapter sequences, such as the MS2 sequence shown in SEQ ID NO: 17.

18. The prime editing system of any one of claims 1-17, wherein the primer binding site sequence is configured to be complementary to at least a portion of the target sequence, preferably, the primer binding site sequence is complementary to at least a portion of the 3′ free single strand of the target sequence caused by the nick in the DNA strand, particularly complementary to the nucleotide sequence at the 3′ end of the 3′ free single strand.

19. The prime editing system of any one of claims 1-18, wherein the RTT sequence is configured to correspond to a sequence downstream of the nick of the target sequence, but contains a desired modification which comprises substitution, deletion and/or addition of one or more nucleosides.

20. The prime editing system of any one of claims 1-19, further comprising a nicking gRNA and/or an expression construct containing a nucleotide sequence encoding the nicking gRNA, the nicking gRNA comprising a leader sequence and a scaffold sequence, the leader sequence is configured to have sufficient sequence identity with the nicking target sequence in the genome, thereby being able to target the CRISPR nuclease or the fusion protein to the nicking target sequence and causing a nick in the nicking target sequence, the nicking target sequence and the editing gRNA target sequence are located on opposite strands of the genomic DNA, and the nick induced by the nicking gRNA and the nick induced by the editing gRNA are about 1 to about 300 nucleotides apart.

21. The prime editing system of any one of claims 1-20, wherein the prime editing system comprises a plurality of editing gRNAs and/or an expression construct containing a nucleotide sequence encoding the plurality of editing gRNAs, correspondingly, the circular RNA comprises multiple sets of RTT-PBS sequences in tandem corresponding to the plurality of editing gRNAs.

22. The prime editing system of claim 21, wherein the plurality of editing gRNAs target different target sequences, and the corresponding sets of RTT-PBS sequences are used to introduce different modifications into the different target sequences.

23. The prime editing system of any one of claims 1-22, wherein the expression construct containing the nucleotide sequence encoding the circular RNA further contains elements required for realizing RNA circularization.

24. The prime editing system of any one of claims 1-23, wherein the “expression construct containing a nucleotide sequence encoding the circular RNA” comprises a coding sequence of the following: 5′-first ribozyme-first circularization arm-RTT-PBS-second circularization arm-second ribozyme-3′, when transcribed into RNA in the cell, the first ribozyme and the second ribozyme can self-cleave to produce “5′-first circularization arm-RTT-PBS-second circularization arm-3”, and the first circularization arm and the second circularization arm can be linked to each other to form a circular RNA containing RTT and PBS.

25. The prime editing system of claim 24, the first ribozyme comprises the nucleotide sequence shown in SEQ ID NO:19, the second ribozyme comprises the nucleotide sequence shown in SEQ ID NO:20, the first circularization arm comprises the nucleotide sequence shown in SEQ ID NO:21, and the second circularization arm comprises the nucleotide sequence shown in SEQ ID NO:22.

26. The prime editing system of any one of claims 1-25, wherein the editing gRNA and the circular RNA and optionally the nicking gRNA are encoded by a same expression construct.

27. The prime editing system of claim 26, wherein the expression construct comprises (e.g., from the 5′ to 3′ direction) a nucleotide sequence encoding the circular RNA, a nucleotide sequence encoding the editing gRNA, and optionally a nucleotide sequence encoding the nicking gRNA.

28. The prime editing system of claim 27, wherein expression of the nucleotide sequence encoding the circular RNA, the nucleotide sequence encoding the editing gRNA, and optionally the nucleotide sequence encoding the nicking gRNA is driven by a same promoter such as the U6 promoter.

29. A method of producing a genetically modified cell, comprising introducing the prime editing system of any one of claims 1 to 28 into at least one of said cell, thereby resulting in modification of the genomic sequence of said at least one cell.

30. The method of claim 29, wherein the cell is from mammals such as human, mouse, rat, monkey, dog, pig, sheep, cattle, cat; poultry such as chicken, duck, goose; plants, including monocots and dicots such as rice, corn, wheat, sorghum, barley, soybean, peanut, Arabidopsis thaliana.
